(19) United States
(12) Patent Application Publication
(10) Pub. No.: US 2025/0210129 A1
(43) Pub. Date: Jun. 26, 2025

(54) Method for Capturing Atomic Details of Proteins Using a 3D Grid for Mutational Analysis

(71) Applicant: KCAT ENZYMATIC PRIVATE LIMITED, Bengaluru (IN)

(72) Inventors: Pravin Kumar R, Bengaluru (IN); Gladstone Sigamani G, Bengaluru (IN); Roopa L, Bengaluru (IN); Likith M, Bengaluru (IN); Anuj J Shetty, Bengaluru (IN)

(73) Assignee: KCAT ENZYMATIC PRIVATE LIMITED, Bengaluru, KA (IN)

(21) Appl. No.: 18/986,755

(22) Filed: Dec. 19, 2024

(30) Foreign Application Priority Data
Dec. 21, 2023 (IN) 202341079086

(51) Int. Cl. G16B 15/30; C12N 9/04; G16B 15/20; G16B 20/50; G16B 40/20

(52) U.S. Cl. CPC G16B 15/30; C12N 9/0006; G16B 15/20; G16B 20/50; G16B 40/20; C12Y 101/01047

(57) ABSTRACT

This invention presents a novel method for engineering glucose dehydrogenase (GDH) proteins by utilizing an atomistic grid-based computational method that analyzes and compares protein atomic compositions. The method involves constructing Localized spherical feature grids (LSFGs) centered around high-energy regions to store atomic properties, enabling comparison with a database of known protein grids. Two comparison techniques are applied: geometric alignment using rotation matrices and quaternions, and transformer-based similarity scoring. High-ranking matches guide functional and stability optimization through mutation design. Unlike conventional methods that require spatial alignment, this approach maps chemical properties directly onto the grid, enabling alignment-free comparisons based on chemical composition allowing comparison of specific protein regions even in the absence of structural similarity. This method provides alignment-free chemical profiling for structurally diverse proteins, facilitating advanced protein engineering and functional annotation.

This instant application contains a Sequence Listing which has been submitted electronically in XML file format and is hereby incorporated by reference in its entirety. Said XML Copy, created on Dec. 11, 2024, is named Seqlisting.xml and is 34000 bytes in size.

### FIELD OF THE INVENTION

This invention relates to the field of bioinformatics, biotechnology, biochemistry, computational biology, molecular biology, Artificial intelligence, Machine-learning

### BACKGROUND OF THE INVENTION

Proteins are functional biomolecules of the cell. As enzymes, they are necessary for the catalysis of chemical reactions. Structural proteins are crucial components of cytoskeleton and locomotory elements of cells. Transporter proteins act as carriers of compounds to different regions of the cell or across membranes. Many proteins are involved in the regulatory mechanism of cells as interactive species in a particular pathway. Proteins can also function as hormones to illicit a desired gene expression or trigger a specific biochemical pathway. Protein function is entirely dependent on the three-dimensional structure of the proteins and the specific physiochemical interaction in between two protein species or protein and ligand species. Protein structures are in turn, defined by the interaction, packing and spatial arrangement of the amino acid residues constituting the long polypeptide sequence of the protein.

Structure-function relationship studies of proteins is a well-studied subject in the field of bioinformatics and molecular biology to determine the function of uncharacterized proteins. Most structure-function relationship studies depend on comparative studies between two or more proteins, where a protein of unknown characteristics is compared using sequence and structural similarity to a protein with defined characteristics. Using this comparison, it can be inferred that two proteins that share similarity in sequence and structure are homologs of each other and thus share a similar function. Furthermore, structural homologs are more prevalent than sequence homologs as protein structures are evolutionarily more conserved that is, two proteins that have low sequence similarity (˜30% sequence identity) can still have conserved domains and structural similarities.

Homology based structural modelling is a method that is used to determine the structure of a protein whose structure was not derived experimentally. In homology modelling, structures are derived using a sequence homology-based search, wherein the 3D structure details of local high identity matching regions of a template structure are used to model the structure of the query protein sequence.

One of the challenges in protein comparison is that when two proteins do not share similar global structures or sequences, the traditional superimposition methods which rely on overall alignment, becomes ineffective (FIG. 1). A grid-based method overcomes that limitation by focusing on the localized atomic composition within the protein rather than its overall structure.

A grid based structural characterization of a protein structures is a commonly used method to derive information of the protein structure using organized grid points to capture atomistic details since grid points are evenly spaced and can provide for regular normalized data points which are easier to compute when compared to the unique, often irregular spatial distribution of atoms in a protein structure. Several grid-based methods to study protein structure and function have been developed.

FEATURE is a tool that was developed to structurally and functionally characterize microenvironments with protein structures. The tool defines the microenvironment by measuring physicochemical properties of atoms around a specific chosen site using concentric shells of 1.25 Å thickness to capture 80 different biochemical characteristics such as charge and polypeptide-based characteristics such as secondary structure type resulting in a numeric vector of length 480. The tool provides unique features of functional sites by using non-site microenvironment characterization to eliminate background properties. (Bagley, S. C., et. al., 1995)

Torng et al. (2017) developed a method for structure-based protein analysis using 3D-convolutional neural networks to predict amino acids most compatible with a specific location within a protein structure. Protein microenvironments are defined as atom channels, one for each atom type (C, O, N, S), within a 20 Å box around a central location within the protein to develop a visualization method known as a “atom importance map”, to inspect individual contributions of each atom within the input. The method was developed based on the principle that mutations introduced into a protein sequence is considered non-detrimental if the newly introduced residue can maintain the critical interactions observed between the wild-type residue and its surrounding residues. Atom importance map visualization provides information to validate and rank introduction of mutations into the protein structure.

Siamese Atomic Surfacelet Network (SASNet) is a tool that was developed to determine the probability of an amino acid on the surface of the protein to interact with another amino acid on the surface of another protein by voxelizing the local atomic environments, or “surfacelets” into 4D grids, the last dimension being the atomic element type. The method uses a Siamese-like three-dimensional convolutional neural network trained on the database of interacting protein structures (DIPS) which leverages already existing protein complex structures in their bound states (Townshend et al., 2019).

Sato et al. (2019) developed a quality assessment method for protein tertiary structure prediction based on deep neural network and three-dimensional convolutional neural network layer by assessing the local residue structure quality and integrating the local residue assessments to derive a whole-structure model quality assessment. Local residue quality assessment was conducted using a 3D grid bounding box centered on the Cα atom of a residue. The bounding box was oriented with respect to the vectors formed between the Cα, C and N atoms of the residue backbone of the structure. The bounding grid was divided into 1 Å voxels and atoms within the voxels were used to characterize the voxels based on atom types which were assigned to an independent channel of the neural network.

The above-mentioned literatures detail tools developed to map the atomic properties of a protein structure. However, a cubic grid with only a six-faced comparison restriction would limit method for spatial orientation-independent comparison of atomic properties. Therefore, in the present invention we report a method that employs localized spherical feature grids to capture atomistic properties as spherical grids can offer many different rotational orientations for increased variations in comparison between regions of proteins.

### PRIOR ART

Torng, W., Altman, R. B. 3D deep convolutional neural networks for amino acid environment similarity analysis. BMC Bioinformatics 18, 302 (2017). https://doi.org/10.1186/s12859-017-1702-0
Bagley, S. C., & Altman, R. B. (1995). Characterizing the microenvironment surrounding protein sites. Protein Science, 4(4), 622-635. https://doi.org/10.1002/pro.5560040404
Townshend, R. J. L., Bedi, R., Suriana, P., & Dror, R. O. (2019). End-to-End learning on 3D protein structure for interface prediction. Neural Information Processing Systems, 32, 15616-15625. https://papers.nips.cc/paper/9695-end-to-end-learning-on-3d-protein-structure-for-interface-prediction.pdf
Sato R, Ishida T (2019) Protein model accuracy estimation based on local structure quality assessment using 3D convolutional neural network. PLOS ONE 14(9): e0221347. https://doi.org/10.1371/journal.pone.0221347
Schwerdtfeger, P., & Nagle, J. K. (2018). 2018 Table of static dipole polarizabilities of the neutral elements in the periodic table*. Molecular Physics, 117(9-12), 1200-1225. https://doi.org/10.1080/00268976.2018.1535143
Zhao, Y. H., Abraham, M. H., & Zissimos, A. M. (2003). Fast Calculation of van der Waals Volume as a Sum of Atomic and Bond Contributions and Its Application to Drug Compounds. The Journal of Organic Chemistry, 68(19), 7368-7373. https://doi.org/10.1021/jo0348080

### OBJECT OF THE INVENTION

The primary objective of the present invention is to provide a method for alignment-free protein comparison at the atomic level, using an atomistic grid match-based computational approach. This method enables the identification of chemical and functional similarities across proteins without relying on traditional structural alignment, thereby overcoming limitations associated with conventional spatial alignment methods. The invention achieves high-resolution chemical profiling by constructing a finely spaced, three-dimensional grid around protein structures, which captures the potential energy landscape across the protein, allowing for the identification of high-energy residues. Another objective of the invention is to analyze and identify high-energy residues and functionally relevant localized regions within the protein structure by capturing the potential energy landscape. This identification facilitates targeted modifications in protein engineering to enhance desired properties. A further aim is to construct the localized spherical feature grid (LSFG) around the identified regions and design protein variants with enhanced functionality and stability, especially focusing on enzymes such as glucose dehydrogenase, where improvements in co-factor recycling and catalytic efficiency are targeted. Additionally, the invention facilitates functional annotation of uncharacterized proteins by comparing their LSFGs to known protein structures within a pre-established database, enabling prediction of potential functions, active sites, binding pockets, and catalytic domains. Another aspect of the invention involves optimizing antibody engineering, using LSFG-based analysis to enhance binding specificity, stability, and affinity for target antigens in therapeutic contexts. Overall, the objectives of this invention advance the field of protein engineering by providing a versatile and precise method for protein analysis, functional prediction, and the development of biologically active proteins for industrial and therapeutic applications.

### SUMMARY OF THE INVENTION

This invention introduces a computational method for engineering proteins, especially a glucose dehydrogenase (GDHs), by analyzing their atomic composition through a 3D grid-based system. Unlike conventional methods that depend on structural alignment, this approach arranges a protein's atoms into a fine, three-dimensional grid to capture potential energies from all atoms in the protein. These properties are then used to identify high-energy residues and functional regions within the protein. The process involves constructing a localized spherical feature grid (LSFG) around targeted regions of the protein to store atomic-level information such as atom type, partial charge, polarity, atomic volume, solvent accessible surface area, electronegativity, ionization energy, polarizability, electron affinity, electrostatic potential, solvent accessibility and coordination number to derive composite values, stored in each grid point for comparison with predefined LSFG's of other protein structures in a comprehensive database. To determine the best match, the invention employs two methods: (1) a geometric alignment method, where rotation matrices and quaternions systematically test all orientations, and (2) a transformer-based similarity approach, which captures spatial and chemical patterns at each grid point. The method is particularly useful for identifying functional motifs, optimizing enzyme stability, and designing mutations for improved protein functionality. This approach was used to engineer glucose dehydrogenase (GDH) variants with enhanced stability and co-factor recycling efficiency, relevant in biocatalytic processes. Additionally, the approach has potential applications in antibody engineering and functional annotation of novel proteins by aligning chemical properties without requiring structural similarity.

### BRIEF DESCRIPTION OF DRAWINGS

FIG. 1: Comparison of structures of Protein of interest (Protein 1) and protein with desired characteristics (Protein 2) using three-dimensional Structural superimposition. A region of the protein where structural superimposition is not effective is shown in the insert.

FIG. 2: Schematic representation of protocols used to identify regions of interest and derive localized spherical feature grids to engineer proteins or identify and characterize domain function.

FIG. 3, wherein FIG. 3A is a Three-dimensional structure of Glucose dehydrogenase (GDH) enzyme, a protein of interest, FIG. 3B shows the energy grid enclosing the whole protein, FIG. 3C is a Close-up view of the grid with grid points, grid spacing and probe atoms, FIG. 3D shows Residue-wise grid energy (kcal·mol⁻¹) contributions.

FIG. 4: wherein FIG. 4A shows Construction of localised spherical feature grid, capturing the atomic information of the residues closer to the grid points. FIG. 4B For two or more atoms closer to a grid point, a composite value of the descriptors is stored, which results in a localized characteristic for each grid point that is unique for a particular region of a protein.

FIG. 4C The grid points are stored in a table that contains all the atomistic descriptors as columns. FIG. 4D Each grid point captures 12 atomistic descriptors. FIG. 4E Atoms equidistant from two grid points, the grid point (G1) which is closer to another atom bonded to the equidistant atom is preferred for calculation of composite parameters.

FIG. 5: Construction of comprehensive database with predefined localized spherical feature grids derived from a protein dataset that contains proteins, defined with either desired characteristics such as thermostability, organic solvent tolerance, pH tolerance or defined functional domains.

FIG. 6: Comparative analysis to identify structural and functional similarities using an arrangement of the atoms in a protein's 3D structure deposited onto a finely spaced spherical grid.

FIG. 7: A process of generating a comprehensive set of rotational orientations for each localized spherical feature grid using rotation metrics and quaternions so that the feature grids can be systematically matched with multiple precomputed grids in the dataset by considering all possible orientations.

FIG. 8: Scoring and ranking comparison between two localized spherical feature grids (LSFGs). FIG. 8A Comparing the query rotated grid's vectors to those of the rotated dataset grid using Euclidean distance for spatial difference or cosine similarity for directional alignment across ‘n’ iterations of LSFG orientations. FIG. 8B A transformer-based approach wherein each grid point is tokenized to capture complex relational data across spatial and chemical dimensions to match, sore and rank LSFGs within a protein.

Table 1: Atomic properties calculated at every grid point.

Table 2: Atomistic property descriptors captured for each grid point for a segment of the localized spherical feature grid.

Table 3: Table shows residue difference relative to SEQ ID No: 1 on engineered GDH.

### DETAILED DESCRIPTION OF THE INVENTION

### Terminologies:

“Protein,” “polypeptide”, and “peptide” are used interchangeably herein to denote a polymer of at least two amino acids covalently linked by an amide bond, regardless of length or post-translational modification.

“Amino acids” are referred to herein by either their commonly known three-letter symbols or by the one-letter symbols recommended by IUPAC-IUB biochemical nomenclature commission.

“Atomistic Grid Match” herein refers to a computational technique arranging protein atoms into a spherical 3D grid to capture atomic-level details for protein comparison and analysis.

“3D Spherical Grid” herein refers to a high-resolution grid enclosing the protein structure, spaced at regular intervals

“Grid points” herein refers to finely spaced positions in the grid, where probe atoms are placed to capture relevant data.

“Probe Atoms” herein refers to atoms (C, O, N, H, S, P) used in the grid to compute potential energy

“Angstrom (Å)” herein refers to a unit of length equal to 0.1 nanometers, used to measure atomic-scale distances

“Potential energy” herein refers to energies which are calculated based on the Coulombic and Lennard-Jones potential functions.

“High-Energy Residues” herein refers to the top 5% of residues with the highest energy values, identified by sorting all residues in descending order based on their energy values.

“Localised region” herein refers to the region around the high-energy residues and the super-secondary structures, domain, or motifs are identified for the protein of interest with unknown function.

“Localized Spherical Feature Grid (LSFG)” herein refers to a spherical grid with a 6 Å radius around localized regions, capturing both chemical and spatial information of the protein of interest.

“Atomic Properties” herein refers to the chemical and physical characteristics of atoms, such as atom type (C, O, N, H, S, P), partial charge, polarity, atomic volume (Å³), accessible surface area (Å²), electronegativity, ionization energy (eV), polarizability (Å³), electron affinity (eV), electrostatic potential (kcal/mol), solvent accessibility, and coordination number

“Composite values” herein are the aggregated representations of multiple atomic properties (such as atom type, partial charge, and polarity) at a specific grid point.

“Solvent Accessible Surface Area (SASA)” herein refers to the area of an atom exposed to the solvent, helping to identify buried or exposed regions in the protein.

“Electronegativity” herein represents an atom's ability to attract electrons.

“Polarizability” herein indicates the flexibility of an atom's electron cloud.

“Coordination Number” herein specifies the number of atoms bonded to a central atom.

“Energy Maps” herein refers to the 2D representation of potential energy distributions around the protein, created using probe atoms.

“One-Hot Encoding” herein refers to a method for representing atom types (e.g., C: [1, 0, 0, 0, 0, 0]) as a vector.

“Self-attention mechanism” herein refers to a technique in transformers that allows the model to weigh the importance of different tokens (grid points) in relation to each other, enabling it to capture both local and global patterns in the data.

“Positional encodings” herein refers to the information added to the input data in transformers to preserve the spatial or sequential positions of elements, ensuring that the model maintains the relative positions or distances within the data.

“Contrastive learning” herein refers to a machine learning technique that trains models by comparing pairs of similar and dissimilar examples, encouraging the model to learn distinct features for each class.

“Similarity scores” herein refers to the values that indicate how similar two grids are to each other, often used in matching or ranking.

“Attention scores” herein refers to the values that quantify how much focus each grid point in a transformer model should give to other grid point based on their relationships.

“Query vector” herein refers to a vector in the transformer model that represents the information the token seeks from others, used to compute attention scores during the self-attention mechanism.

“Key vector” herein refers to a vector in the transformer model that represents the information offered by a token, used to match with the query vector in the self-attention mechanism.

“Value vector” herein refers to a vector that holds the actual information of a token, which is weighted by the attention score during the self-attention mechanism.

“Amino acid difference or residue difference” refers to a change in the residue at a specified position of a polypeptide sequence when compared to a reference sequence.

This invention provides a novel method for engineering proteins, specifically glucose dehydrogenase, by utilizing an atomistic grid match based computational method to analyze and compare the atomic composition of proteins. The method arranges the atoms in a protein's 3D structure into a finely spaced three-dimensional spherical grid. This spherical grid captures atomic-level details in a highly localized manner, allowing for the comparison of specific regions within two proteins, even if they are globally dissimilar.

In conventional protein comparison methods, spatial alignment is heavily relied upon to superimpose proteins or binding sites to reveal conformational similarities. However, these approaches are limited when proteins lack significant structural similarity, even though they may possess similar chemical environments in functionally relevant regions. This invention provides an alternative by mapping chemical properties directly onto a 3D grid, allowing for alignment-free comparison focused on chemical composition rather than spatial arrangement.

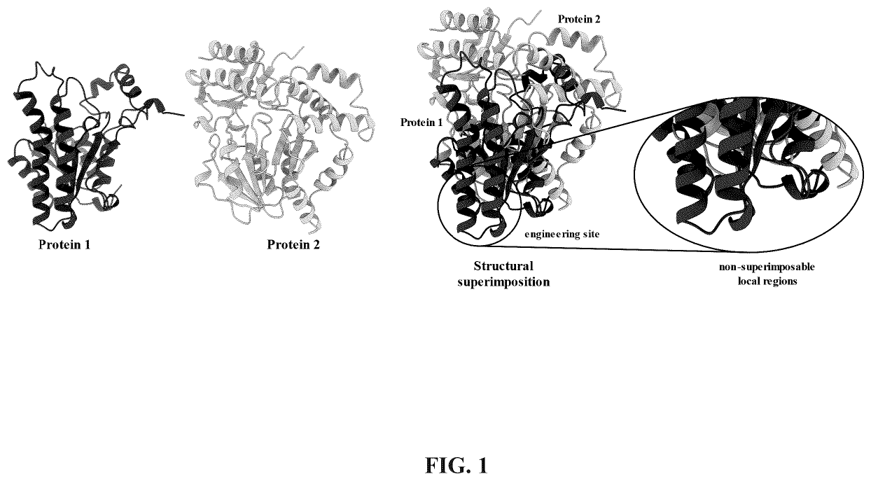

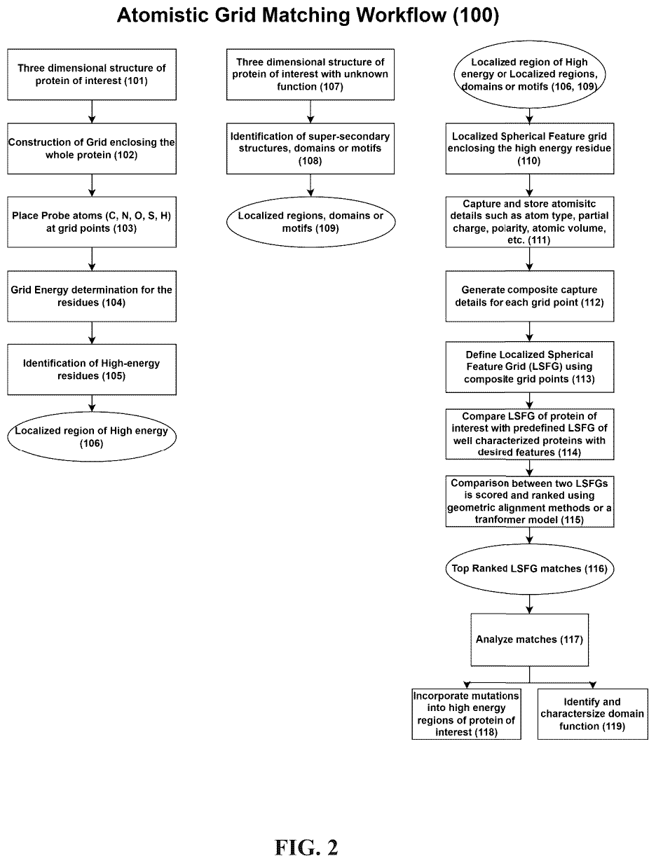

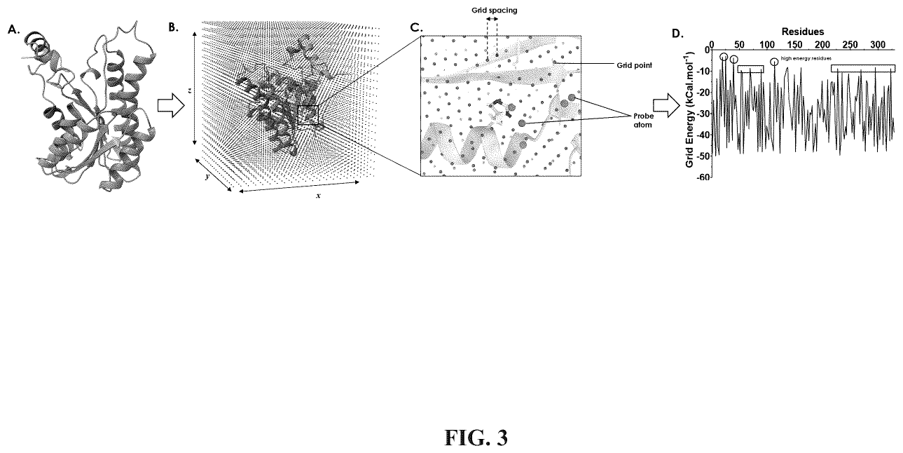

FIG. 1 shows the two proteins with different overall structures which are non-superimposed using structural alignment. FIG. 2 shows the Atomistic Grid matching workflow (100). The first step involves building a three-dimensional grid around the protein of interest (101) based on its X-ray crystallographic structure (FIG. 3A). The construction of grid (102) encloses the entire protein ensuring high-resolution coverage (FIG. 3B). The grid is composed of grid points that are spaced at regular intervals of 1 Å (angstroms). Each grid point captures the potential energy at each atom and position of surrounding atoms. At each grid point, probe atoms (103) such as carbon, nitrogen, oxygen, sulfur, phosphorous, and hydrogen are positioned to analyze the potential energy landscape around the protein's structure. These probe atoms help capture the potential energies associated with the interactions between the protein's atoms and the probe atoms. This step allows for the creation of a detailed energy map around the protein (FIG. 3C). Once the potential energies have been calculated at each grid point, the method sums the energies of all probe atoms near each residue in the protein to determine the grid energy of each residue (104). By analyzing these summed values and ranking them in descending order, top 5% of the residues with high energy values are termed as “High-energy residues” (105) and region around these high-energy residues are considered high-energy localized regions (106) and are targeted for further engineering (FIG. 3D). On the other hand, the super-secondary structures, domain, or motifs are identified (108) for the protein of interest with unknown function (107) are considered as localised regions (109). After identifying the localised regions (106, 109), a 6 Å localized spherical feature grid (110) is constructed around each localised region to capture and store the atomistic details, centered on the amino acid, encompassing both the chemical and spatial information in that local region. The choice of a 6 Å radius ensures that all nearby atoms contributing to chemical interactions (e.g., hydrogen bonds, hydrophobic patches, etc.) are included.

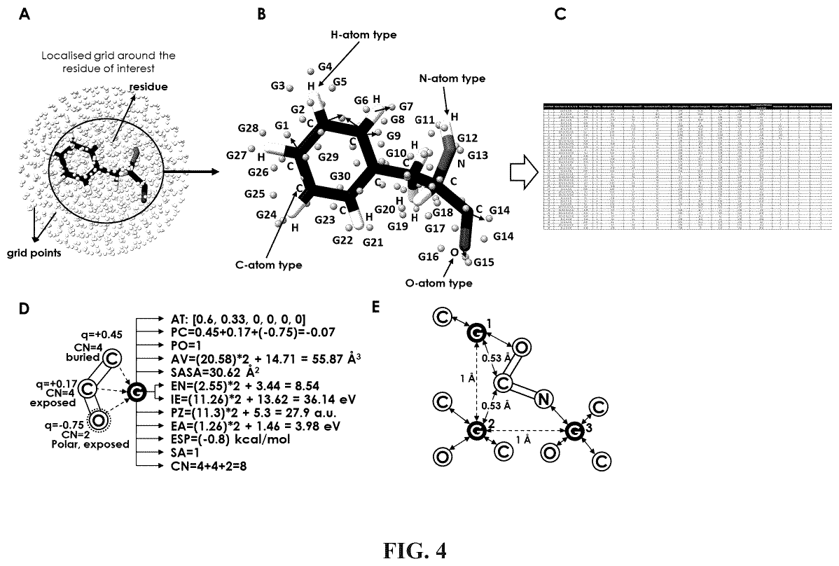

FIG. 4A explains the construction of localised spherical feature grid, the grid points in the grid build around the localised region captures the atomic details of the residues. The localised spherical grid is a uniformly spaced grid, where grid points are spaced 1.0 Å apart. The grid points are assigned C, O, N, S, H, and P atom types based on the closer atom (FIG. 4B) where C is Carbon atom-type, O is Oxygen atom-type, N is Nitrogen atom-type, S is Sulphur atom-type, His Hydrogen atom-type, and P is phosphorous atom-type. Each grid point that intersects or overlaps with an atom captures and stores (111) several atomic properties, including atom type, partial charge, polarity, atomic volume, solvent accessible surface area, electronegativity, ionization energy, polarizability, electron affinity, electrostatic potential, solvent accessibility, and coordination number as described in Table 1 (FIG. 4C, Table 2). This approach of assigning atom types to grid points is distinct from the traditional method of placing a probe at each grid point for calculations.

The Atom Type (AT) identifies each atom as Carbon (C), Nitrogen (N), Oxygen (O), Hydrogen (H), Sulphur(S), or phosphorous (P) and is typically encoded as a categorical or one-hot vector (e.g., C: [1, 0, 0, 0, 0, 0]). Partial Charge (PC) reflects the charge distribution based on the atom's bonding environment and is represented as a real number derived from molecular mechanics or quantum calculations (e.g., C: +0.1, O: −0.8, etc.). Polarity (PO) is a binary indicator of whether the atom is polar or non-polar, where polar atoms (like Oxygen, nitrogen, sulphur and phosphorous) are assigned a 1 and non-polar atoms (like Carbon) are assigned a 0. Atomic Volume (AV) represents the approximate space occupied by an atom (e.g., 20.58 Å³ for Carbon), and Solvent Accessible Surface Area (SASA) indicates how much of an atom's surface is exposed to the solvent, with values ranging from 5-10 Å² for Cα and 12-18 Å² for O atoms etc. Electronegativity (EN) shows each atom's ability to attract electrons, which influences molecular bonding; for instance, Carbon has a value of 2.55 and Oxygen 3.44, etc., Ionization Energy (IE) represents the energy required to remove an electron, relevant for chemical reactivity, with Carbon at 11.26 eV and Oxygen at 13.62 eV, etc., Polarizability (PZ) describes the flexibility of an atom's electron cloud, influencing van der Waals interactions (C: 11.3 a.u., N: 7.4 a.u., etc., where a.u. is atomic units). Electron Affinity (EA) indicates the energy change when an electron is added, showing the atom's propensity to gain electrons, with Carbon at 1.26 eV and Oxygen at 1.46 eV etc., Electrostatic Potential (ESP) represents the potential energy of a unit positive charge near an atom, calculated in context and influenced by surrounding atoms.

Solvent Accessibility (SA) is a binary indicator of exposure to solvent (1 for exposed, 0 for buried). Coordination Number (CN) specifies the number of atoms bonded to the central atom, relevant in structural modeling.

When multiple atoms overlap at a single grid point, composite values (112) for the atomic properties are calculated using methods like averaging or weighted selection to reflect the most chemically relevant atom. This ensures an accurate representation of overlapping atomic contributions in a grid point's chemical profile. To derive composite values (112) when multiple atoms overlap at a single grid point, each property must be aggregated to represent the combined effect of these atoms as depicted in the FIG. 4D.

The following provides a stepwise approach to determining a composite property value:
1. Atom Type: For atom type, which is represented as a one-hot encoded vector that denotes a particular element in the format: [C, O, N, H, S, P]. For e.g., Carbon as [1, 0, 0, 0, 0, 0], Oxygen as [0, 1, 0, 0, 0, 0], Nitrogen as [0, 0, 1, 0, 0, 0], hydrogen as [0, 0, 0, 1, 0, 0], sulphur as [0, 0, 0, 0, 1, 0], and phosphorous as [0, 0, 0, 0, 0, 1]. The composite value is obtained by averaging the one-hot vectors of all contributing atoms at a grid point. For instance, if two Carbon and Oxygen overlap, their one-hot vectors are averaged to yield a weighted vector [0.66, 0.33, 0, 0, 0, 0], indicating a contribution of both atom types.
2. Partial Charge Calculation: In the case of partial charge, the composite value is calculated by summing the partial charges of the overlapping atoms. For example, if a Carbon atom has a partial charge of +0.1 and an Oxygen atom has-0.8, the composite charge at the grid point is calculated as (−0.8+0.1)=−0.7.
3. Polarity Determination: Polarity, being a binary attribute, is represented by a value of 1 for polar atoms and 0 for non-polar atoms. To ascertain the composite polarity at a grid point, a logical OR operation is performed on the polarity values of overlapping atoms. Where any atom contributing to the grid point is polar, the resultant polarity value for the grid point is set to 1. For instance, in a case where Carbon (non-polar, polarity=0) and Oxygen (polar, polarity=1) are overlapping, the composite polarity would be set to 1, indicating a polar environment
4. Atomic volume: Each property value is summed across all contributing atoms, considering their individual contributions at the grid point. For instance, if Carbon and Oxygen contribute atomic volumes of 20.58 Å³ and 14.71 Å³, respectively, the composite atomic volume is (20.58+14.71)=35.29 Å³. This method ensures that the final value reflects the combined spatial and chemical characteristics of the grid point.
5. Solvent Accessible Surface Area: Each SASA value of the overlapping atom is summed across, considering their individual contributions at the grid point. The SASA value of each atom is determined by the immediate environment of that atom.
6. Electronegativity: Each property value is summed across all contributing atoms, considering their individual contributions at the grid point. For instance, if Carbon and oxygen contribute electronegativity values of 2.55 and 3.44, respectively, the composite electronegativity value is (2.55+3.44)=5.99. This method ensures that the final value reflects the combined spatial and chemical characteristics of the grid point.
7. Ionization Energy: Each property value is summed across all contributing atoms, considering their individual contributions at the grid point. For instance, if Carbon and oxygen contribute ionization energy values of 11.26 eV and 13.62 eV, respectively, the composite ionization energy value is (11.26+13.62)=24.88 eV. This method ensures that the final value reflects the combined spatial and chemical characteristics of the grid point.
8. Polarizability: Each property value is summed across all contributing atoms, considering their individual contributions at the grid point. For instance, if Carbon and oxygen contribute polarizability values of 11.3 a.u. and 5.3 a.u., respectively, the composite electronegativity value is (11.3+5.3)=16.6 a.u. This method ensures that the final value reflects the combined spatial and chemical characteristics of the grid point.
9. Electron Affinity: Each property value is summed across all contributing atoms, considering their individual contributions at the grid point. For instance, if Carbon and oxygen contribute electronegativity values of 1.26 eV and 1.46 eV, respectively, the composite electronegativity value is (1.26+1.46)=2.72 eV. This method ensures that the final value reflects the combined spatial and chemical characteristics of the grid point.
10. Electrostatic Potential: The value depends on the spatial and environmental context; the composite value is determined based on the direct value of electrostatic potential calculation of all atoms overlapping with a particular grid point.
11. Solvent Accessibility: Solvent accessibility, being a binary attribute, is represented by a value of 1 for exposed atoms and 0 for buried atoms. To ascertain the composite solvent accessibility at a grid point, a logical OR operation is performed on the solvent accessibility values of overlapping atoms. Where any atom contributing to the grid point is exposed, the resultant polarity value for the grid point is set to 1. For instance, in a case where Carbon (buried, SA=0) and Oxygen (exposed, SA=1) are overlapping, the composite SA would be set to 1, indicating an exposed environment.
12. Coordination Number Each property value is summed across all contributing atoms, considering their individual contributions at the grid point. For instance, to all atoms overlapping a grid point “G”, if the number of bonded atoms to a carbon is 4, another carbon is 3, an oxygen is 2 and a nitrogen is 3, then the composite of coordination number is (4+3+2+3)=12. This method ensures that the final value reflects the combined environmental properties of the atoms that overlap a single grid point

For an atom equidistant from two grid points G1 or G2, the preferred grid point for composite parameter calculation is selected from the grid point to which another atom is selected for composite parameter calculation and that another atom is bonded or attached to the atom equidistant from the grid points (FIG. 4E).

The resultant grid point vector integrates the combined chemical properties of the overlapping atoms, as in the following example: [0.6,0.3,0,0,0,0,−0.07,1,55.87,30.32,8.54,36.14,27.9,3.98,−0.8,1,8]. This vector structure serves to represent the composite effect of all contributing atoms at a specific grid point.

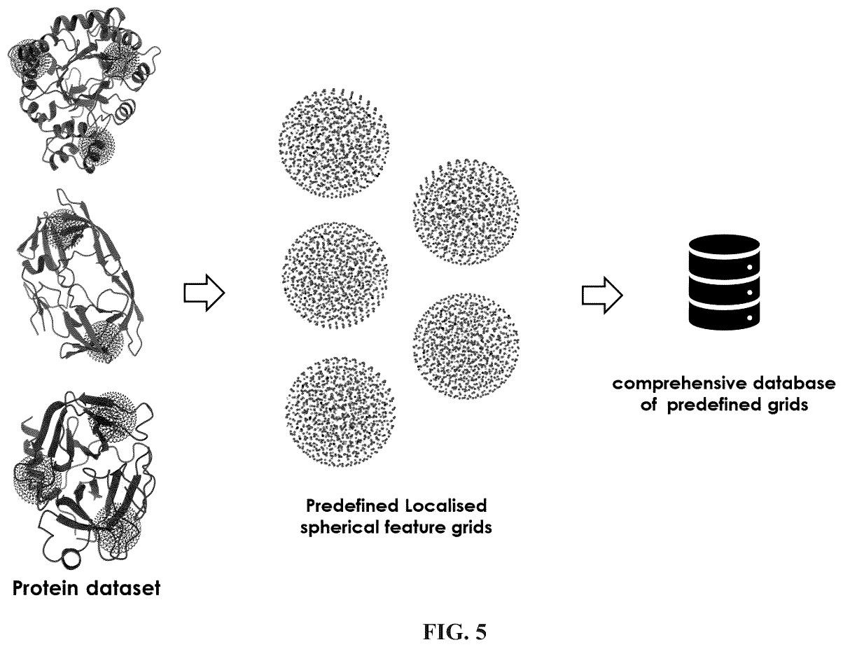

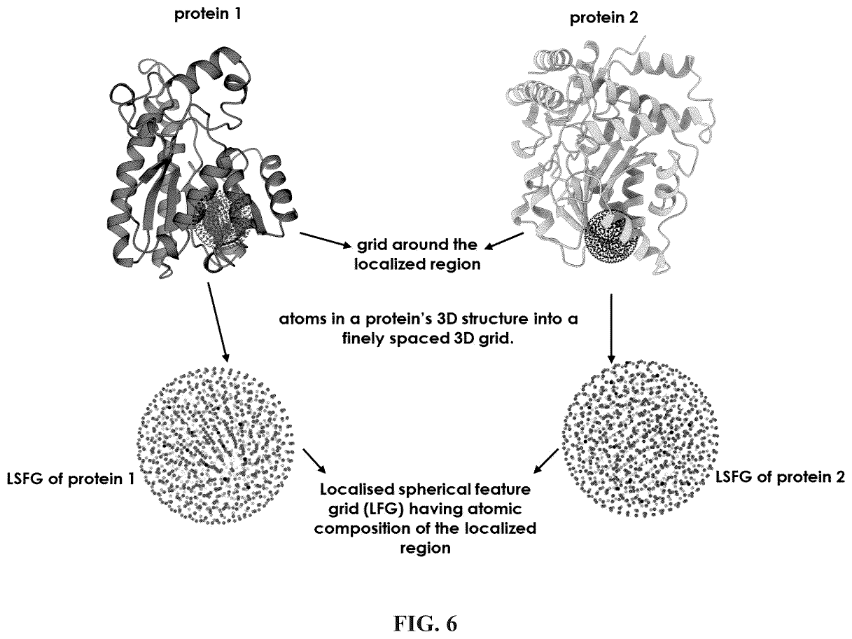

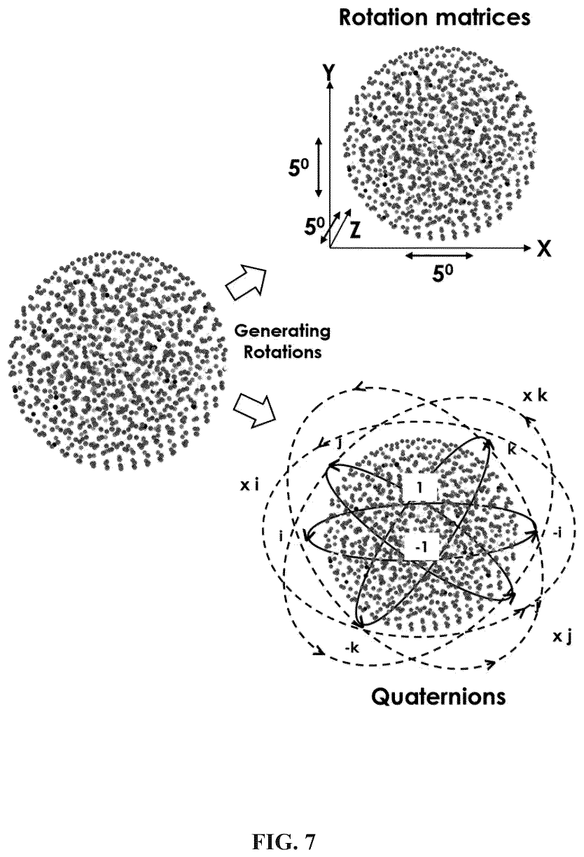

The entire localized spherical feature grid is constructed using the vectors at each grid point, which are derived from the composite values calculated for atomic properties (113). For instance, the vectors at individual grid points might appear as follows: Grid point 1: [0.5,0.5,0,0,0,0,−0.35, 1,36.18, . . . ], Grid point 2: [1,0,0,0,0,0,+0.1,0,20.58, . . . ], Grid point 3: [0,1,0,0,0,0,−0.6,1,14.71, . . . ] and so forth. The generated localized spherical feature grid (LSFG) around the protein region of interest is then compared to a comprehensive database of similar predefined grids (114). This database was created from protein datasets, including BRENDA, ProThermDB, ThermoMutDB, FireProt, and an in-house collection of thermally stable enzymes collected from published literatures (FIG. 5). It captures the spatial arrangement of atomic types in regions with similar grid point vectors, allowing for a comparative analysis to identify structural and functional similarities (FIG. 6).

The localised spherical feature grids are compared using two different ways:
1. Geometric Alignment Method (115): The localized spherical feature grid for a protein region of interest can be systematically matched with multiple precomputed grids in the dataset by considering all possible orientations using both rotation matrices and quaternions (FIG. 7). In practice, this process involves generating a comprehensive set of rotational orientations for each grid in the dataset.

Rotation matrices are applied incrementally around each principal axis (X, Y, Z), typically in small increments, such as 5° or 10°, to ensure thorough coverage of potential orientations.

Alternatively, quaternions can be employed to represent rotations in a more efficient manner. Quaternions enable smooth, continuous rotation by defining the rotation as

q=w+xi+yj+zk, where q is applied to each vector at the grid points to rotate it in 3D space.

A quaternion rotation can be applied by calculating v′=qvq⁻¹ where q is the quaternion, v is the vector to be rotated, and v′ is the rotated vector. By systematically varying the quaternion, you can smoothly rotate the grid around any arbitrary axis.

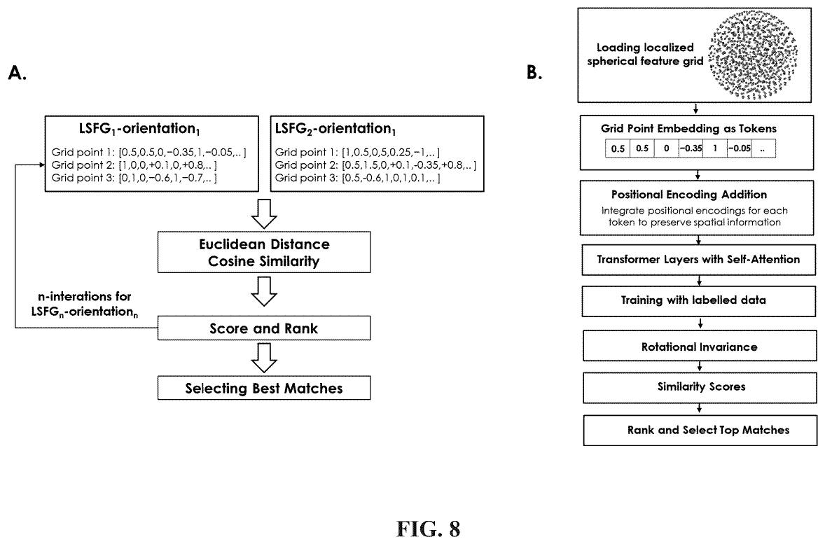

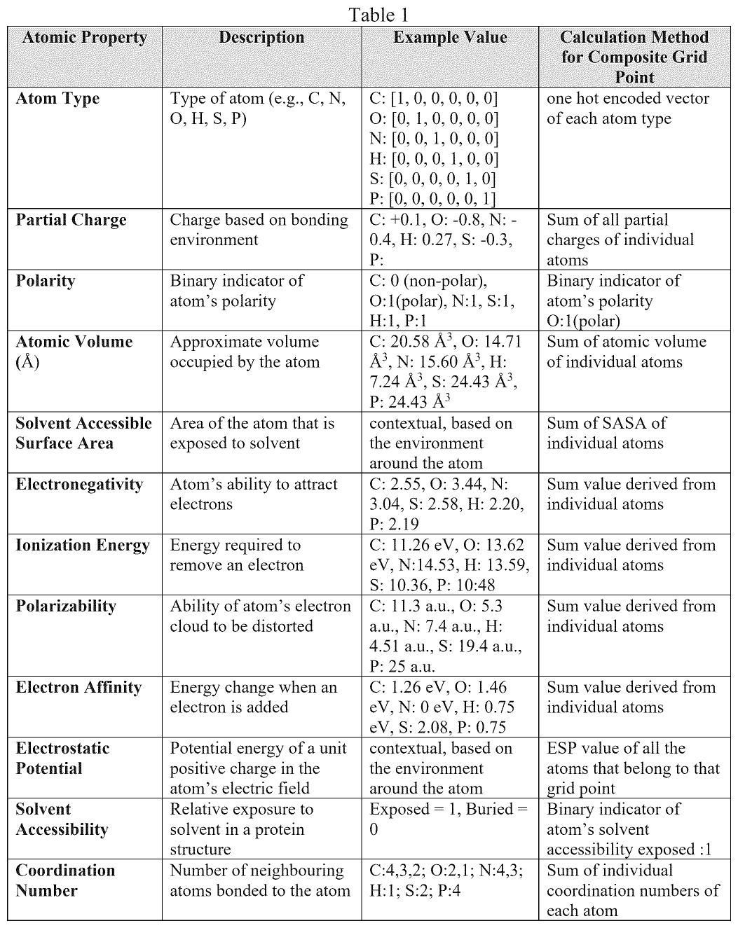

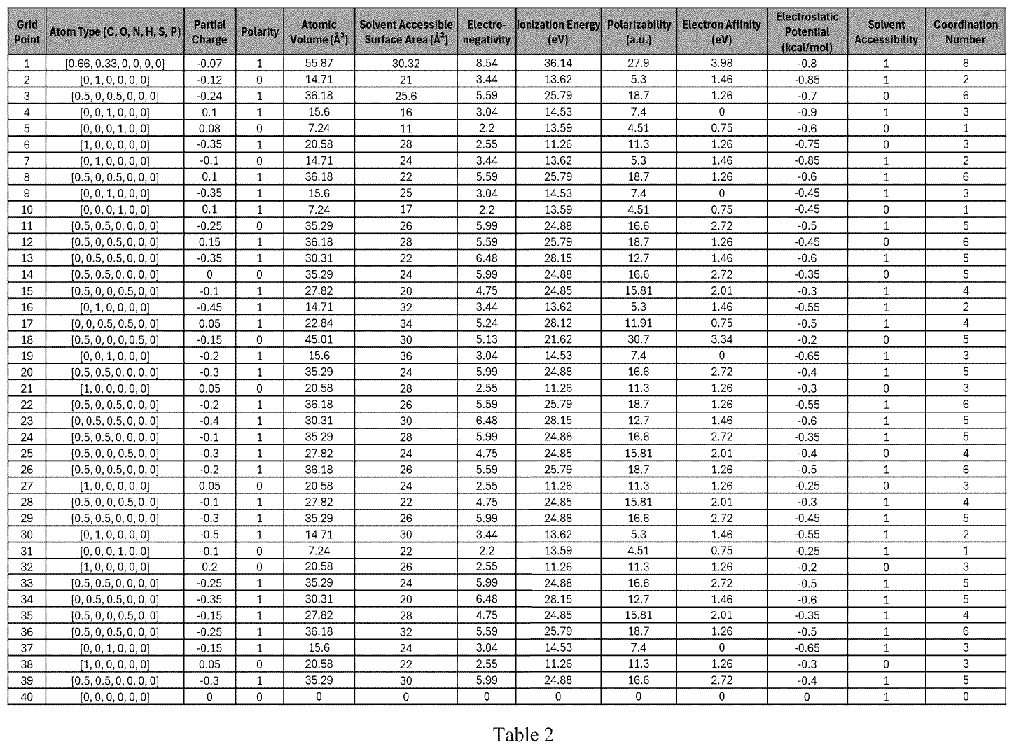

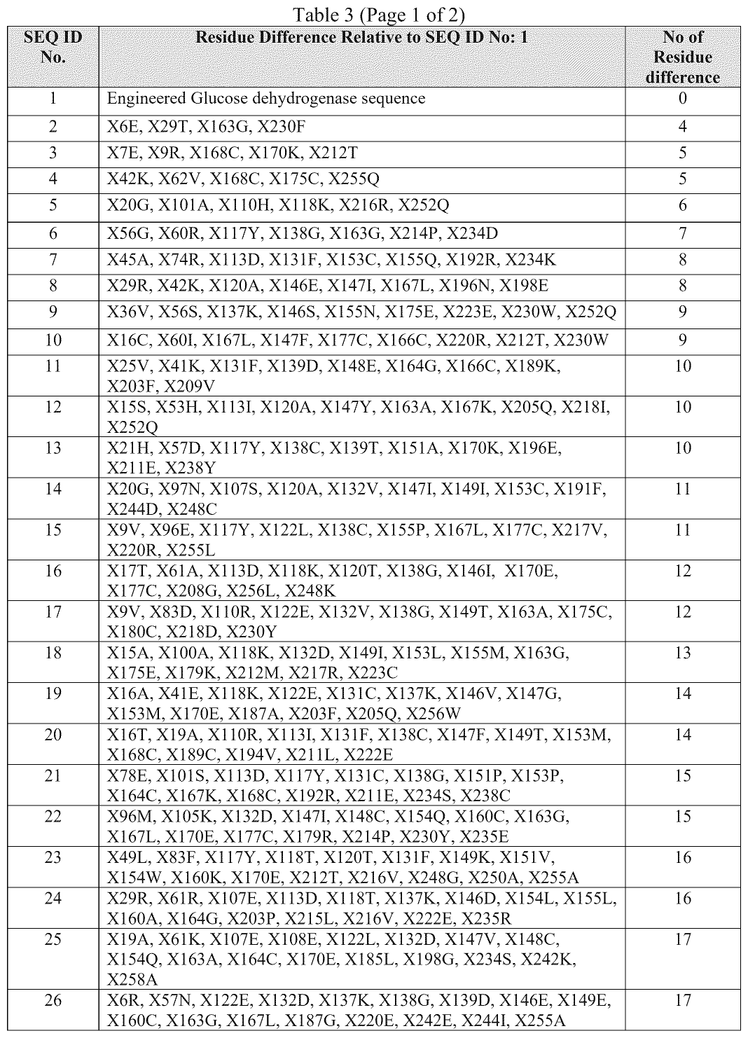

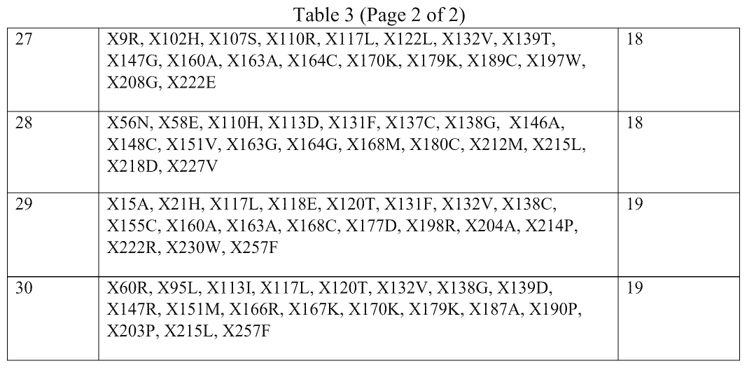

For each rotational orientation, whether derived through rotation matrices or quaternions, a match score is computed by comparing the query rotated grid's vectors to those of the rotated dataset grid. This is achieved by calculating either the Euclidean distance, which reflects the spatial difference in position, or cosine similarity, which assesses directional alignment (FIG. 8A).

Euclidean Distance: Calculate the Euclidean distance between the vector at each grid point in the query grid and the corresponding grid points in each dataset grid. Smaller distances indicate higher similarity.

Euclidean distance between two grid points is given by the following equation:

$$d ( GP 1 , GP 2 ) = ( {\sum }_{i = 1}^{17} \sqrt{( G P {2}_{i}^{2} - G P {1}_{i}^{2} )} )$$

Where, GP1ᵢ and GP2ᵢ are the individual components such as the values of the atomic descriptors, of the two grid point vectors GP1 and GP2

For instance, two grid points, GP1=[0.6,0.3,0,0,0,0,−0.07,1,55.87,30.32, 8.54,36.14,27.9,3.98,−0.8,1,8] of LSFG1, and GP2=[0,1,0,0,0,0,−0.12,0,14.71,21,3.44,13.62,5.3, 1.46,−0.85,1,2,62.56] of LSFG2, the Euclidean distance, d(GP1,GP2) would be calculated as: 53.56

Cosine Similarity: Compute cosine similarity (Sc) between the vectors at each grid point in the query and dataset grids. Cosine similarity falls within the values of (−1,1), wherein, the values of S_{c}=1 indicates that the two vectors are in the same direction, S_{c}=0, indicates that the two vectors are orthogonal and S_{c}=−1, indicates the two vectors are in opposite directions.

Cosine similarity between two grid points is given by the following equation:

$${S}_{c} ( \vec{GP 1} , \vec{GP 2} ) = \frac{{\sum }_{i = 1}^{17} G P {1}_{i} . {GP2}_{i}}{\sqrt{{\sum }_{i = 1}^{17} G P {1}_{i}^{2}} \sqrt{{\sum }_{i = 1}^{17} G P {2}_{i}^{2}}}$$

Where, GP1ᵢ and GP2ᵢ are the individual components such as the values of the atomic descriptors, of the two grid point vectors GP1 and GP2

For instance, two grid points, GP1=[0.6,0.3,0,0,0,0,−0.07, 1,55.87,30.32, 8.54,36.14,27.9,3.98,−0.8,1,8], of LSFG₁ and GP2=[0,1,0,0,0,0,−0.12,0,14.71,21,3.44,13.62,5.3,1.46,−0.85,1,2,62.56] of LSFG₂, the Cosine similarity, S_{c}(GP1,GP2) would be calculated as: 0.91, indicating that the vectors are in the same direction

For the comparison between two LSFGs, the combined score, as a function of Euclidean distance and cosine similarity, is given by the following equation:

$$CS ( {LSFG}_{1} , {LSFG}_{2} ) = ( \frac{1}{\sum {w}_{1} . {L}_{1} d ( G P 1 , G P n )} ) + ( \sum {w}_{2} . {L}_{I} {S}_{c} ( \vec{G P 1} , \vec{G P N} ) )$$

Where, w₁ and w₂ are weights derived from the range of Euclidean distances and cosine similarities, respectively, for each grid point compared between LSFG₁ and LSFG₂; L₁d(GP1,GPn) and L₁Sc(GP1,GPn) are the Euclidean distances and cosine similarities, respectively, derived from the comparisons between the normalized vectors GP1, GPn.

Among the various orientations tested, the orientation yielding the highest match score is selected as the best alignment for that dataset grid.

Afterward, all dataset grids are ranked based on their optimal match scores, with the highest-ranking grids representing the closest spatial and chemical alignment with the region of interest. This approach ensures that the dataset grids are compared comprehensively in all possible orientations, with thresholding applied if necessary to retain only grids with significant similarity, thus identifying the most relevant spatial matches across the dataset.

Transformer-Based Similarity Method (115): A transformer-based approach can effectively match, score, and rank localized spherical feature grids within a protein by leveraging its ability to capture complex relational data across spatial and chemical dimensions (FIG. 8B). In this method, each grid point is treated as a token, embedding its atomic and spatial properties (e.g., atom type, partial charge, polarity, etc.), while positional encodings capture 3D coordinates or relative distances to preserve spatial structure. The self-attention mechanism then allows each grid point to interact with others, capturing both local and global spatial patterns across the region. Training on large datasets of labelled grids enables the transformer to recognize patterns that signify similarity, using contrastive learning to maximize the match for similar grids and minimize it for non-matching ones. The model is then fine-tuned for real-time comparisons, generating similarity scores between a query grid and dataset grids, with the highest scores indicating closest matches. Importantly, transformers allow interpretability through attention scores, which highlight key atomic features contributing to similarity, aiding in ranking. These attention scores quantify the relevance of one token (or grid point) to another within a sequence or spatial grid, enabling the model to focus on important features and relationships. The attention mechanism is central to transformers, as it calculates how much influence each grid point has on others by computing a score based on the interactions between their feature vectors. This score is derived using the Query (Q), Key (K), and Value (V) vectors for each token, where the Query vector represents the information the token seeks, the Key vector represents the information the token offers, and the Value vector contains the actual information. For each grid point iii, the Query and Key vectors are calculated as:

$$Q i = {W}_{Q} \cdot {x}_{i} , {K}_{i} = {W}_{K} \cdot {x}_{i} , {V}_{i} = {W}_{V} \cdot {x}_{i}$$

where W_{Q}, W_{K}, and W_{V} are learnable matrices and xi is the feature vector of grid point i.

The attention score between two grid points i and j is computed as the dot product of their Query and Key vectors, scaled by the square root of the key dimension dₖ and this score indicates how much token j's features should contribute to token i's representation.

$${Score}_{ij} = \frac{{Q}_{i} . {K}_{j}}{\sqrt{{d}_{k}}}$$

The raw scores are then normalized using the SoftMax function to ensure they sum to 1:

$${α}_{i j} = softmax ( {Score}_{i j} ) = \frac{exp ( {Score}_{i j} )}{{\sum }_{k} exp ( {Score}_{i j} )}$$

The result, αᵢⱼ, represents the normalized attention score that reflects the influence of grid point j on grid point i. These attention scores are used to compute a weighted sum of the Value vectors across all grid points j, updating the representation of grid point i:

$${Z}_{i} = {\sum }_{j} {α}_{i j} . {V}_{j}$$

where zᵢ is the updated feature representation of grid point i, incorporating the contributions of all other grid points weighted by their attention scores.

The attention scores help the model to capture both local and global spatial relationships between grid points based on their chemical and spatial features. This enables the transformer to prioritize more relevant grid points during similarity ranking.

For instance, considering two grid points, GP1=[0.6,0.3,0,0,0,0,−0.07,1,55.87,30.32, 8.54,36.14,27.9,3.98,−0.8,1,8], of LSFG₁ and GP2=[0,1,0,0,0,0,−0.12,0,14.71,21,3.44,13.62,5.3,1.46,−0.85,1,2,62.56] of LSFG₂, the attention scoring is as follows:

### The Feature Vectors

$$GP 1 : {X}_{G P 1} = [ 0.6 , 0.3 , 0 , 0 , 0 , 0 , - 0 \text{.07} , 1 , 55.87 , 30.32 , 8.54 , 36.14 , 27.9 , 3.98 , - 0.8 , 1 , 8 ]$$

$$GP 2 : {X}_{G P 2} = [ 0 , 1 , 0 , 0 , 0 , 0 , - 0.12 , 0 , 14.71 , 21 , 3.44 , 13.62 , 5.3 , 1.45 , - 0 \text{.85} , 1 , 2 ]$$

### Query (Q), Key (K), and Value (V) Vectors

Assuming Weight matrices W_{Q}, W_{K}, W_{V} are identity matrices for simplicity:

$$1. {Q}_{G 1} = {W}_{Q} \cdot {X}_{G 1} , {K}_{G 2} = {W}_{K} \cdot {X}_{G 2} , {V}_{G 2} = {W}_{V} \cdot {X}_{G 2}$$

### Attention Score (Scoreᵢⱼ)

$${Score}_{i j} = \frac{{Q}_{i} . {K}_{j}}{\sqrt{{d}_{k}}}$$

$${Q}_{G 1} . {K}_{G 2} = ( 0 \text{.66} \cdot 0 ) + ( 0 \text{.33} \cdot 1 ) + ( 0 \cdot 0 ) + ( 0 \cdot 0 ) + ( - 0 \text{.07} \cdot - 0. 12 ) + ( 1 \cdot 0 ) + ( 55.87 \cdot 14.71 ) + ( 30 \text{.32} \cdot 21 ) + ( 8 \text{.54} \cdot 3.44 ) + ( 36 \text{.14} \cdot 13.62 ) + ( 2 \; 7.9 \cdot 5.3 ) + ( 3.98 \cdot 1.46 ) + ( - 0.8 \cdot - 0. 85 ) + ( 1 \cdot 1 ) + ( 8 \cdot 2 ) = 2136.71$$

Assuming dₖ=17 (Feature Vector Length)

$${Score}_{i j} = \frac{2 136.71}{\sqrt{17}} \approx 518.2$$

### Softmax Normalisation

Assuming we compare GP1 with GP2, GP3 and GP4, the scores are

$${Score}_{i j} = 518.2 , {Score}_{13} = 490 , {Score}_{14} = 460$$

$$exp ( {Score}_{12} ) = exp ( 518.2 ) \approx 2.1 \times {10}^{225}$$

$$exp ( {Score}_{13} ) \approx 6.3 \times 1 {0}^{212} ,$$

$$exp ( {Score}_{14} ) \approx 4.4 \times 1 {0}^{200}$$

$${α}_{i j} = softmax ( {Score}_{i j} ) = \frac{exp ( {Score}_{i j} )}{{\sum }_{k} exp ( {Score}_{i j} )}$$

$${α}_{12} = 1 ; {α}_{13} \approx 3.99 \times {10}^{- 12} ; {α}_{14} = 5.64 \times {10}^{- 25}$$

The attention score for GP2 (α₁₂=1) with respect to GP1 is dominant and is the highest ranked followed by GP3 and GP4.

To address rotational variance, data augmentation with random rotations can be applied during training, or rotationally invariant transformers can be used to handle orientation differences directly.

The top-ranked Localized Spherical Feature Grid (LSFG) matches are analyzed to gain insights into the protein structure-function relationship. This analysis involves several steps, with a focus on incorporating mutations into the protein of interest and identifying key functional domains.

Analysis of Top-Ranked LSFG Matches: Once the LSFGs from the protein of interest are compared with the LSFGs in the dataset (using the two methods outlined previously), the highest-ranked matches are selected. These high-ranking LSFGs represent grid regions in the protein that exhibit the most similarity to known protein regions with well-characterized functions. By analyzing the atomic-level features in these matched regions (such as atom types, charges, hydrophobicity, and spatial arrangement), it is possible to identify conserved patterns and functional motifs shared between the protein of interest and known functional protein domains.

Incorporation of Mutations: The information derived from the top-ranked matches can be used to introduce mutations into the protein of interest. By incorporating specific mutations into the protein's amino acid sequence and observing how they affect the LSFG or the spatial arrangement of atomic properties, it can be predicted how these mutations impact the protein's stability, function, or interactions. If the mutation disrupts a functionally important region, the LSFG comparison can reveal potential compensatory mutations or guide the design of mutations that enhance the desired function.

Characterization of Domain Function: LSFG matching helps in identifying functional domains within the protein of interest. Functional domains are regions of the protein that are responsible for carrying out specific biological activities, such as binding to substrates or interacting with other proteins. By comparing the LSFG of the protein of interest with the LSFGs of known functional domains from the dataset, researchers can identify regions of high similarity that likely correspond to similar functions. The matched regions can be further analyzed to characterize the specific type of function and understand how mutations might influence these activities.

Mapping Mutations to Functional Impacts: Through this analysis, it becomes possible to predict how the mutations could alter the protein's overall function. For example, mutations that occur within regions matching known active sites or interaction domains can be evaluated for their potential to enhance or inhibit enzymatic activity, change binding specificity, or affect protein stability.

After introducing the mutations into the enzyme, the structural integrity and stability of the engineered enzyme are validated using AlphaFold, to predict the modified protein's conformation, ensuring that the introduced mutations do not negatively impact the enzyme's functional integrity. Once the structure is validated, the engineered enzyme gene is cloned into an appropriate expression vector, and the recombinant enzyme is expressed in a suitable host organism. Following expression, the enzyme activity is assessed by testing its catalytic efficiency. This ensures that the engineered enzyme demonstrates the desired improved performance for the intended applications.

This method offers an alignment-free comparison that identifies chemical similarities across structurally diverse proteins, facilitates high-resolution localized chemical profiling, and enhances functional insight into protein interactions.

In some embodiments, the antibodies are engineered using this method. This approach can be applied to engineer antibodies with enhanced binding specificity, stability, and affinity for their target antigens. By understanding how mutations in key functional regions affect antibody structure and interaction, this method can be used to optimize antibody properties for therapeutic use, such as in cancer immunotherapy, autoimmune disease treatments, or infectious disease management. Furthermore, this method can be used to identify the functionality of a protein by analyzing the spatial arrangement of atomic types within its functional domains. By matching the LSFGs of the protein of interest with those in a database of known protein structures with defined structure function characteristics, we can predict the function of uncharacterized proteins and identify novel functions such as enzyme activation loops of tyrosine kinases, TATA box binding proteins, nuclear localizing signals and SH3 binding domains. This is particularly valuable for the functional annotation of novel proteins, allowing for the identification of active sites, binding pockets, or catalytic domains.

The local spherical feature grid of the present invention was used to engineer and design variants of a Glucose dehydrogenase (GDH) enzyme for improved functionality and co-factor recycling ability. Enzymes such as short-chain dehydrogenase/reductase, imine reductases, reductive aminases, amine-dehydrogenases, amino-acid dehydrogenases, ene-reductase and other oxidoreductase enzymes bind Nicotinamide adenine dinucleotide phosphate (NAD(P)H) molecules as cofactors for a source of hydrides required during reduction reactions. GDH, therefore, is an enzyme of immense utility in biocatalysis for the replenishment of NAD(P)H cofactor that is consumed during reduction reactions. GDH enzymes are coupled with any reductase enzyme in a one-pot reaction with a sacrificial substrate such as glucose to convert oxidized NAD(P)⁺ to reduced NAD(P)H. Hence, another objective of the current invention is to use the method of the localized spherical feature grids described in the present invention to design variants through enzyme engineering for achieving an improvement in GDH stability and recycling efficiency.

Specifically, the present invention provides for an engineered glucose dehydrogenase designed using the localized spherical feature grid method descried in the present invention and the glucose dehydrogenase shows 90% sequence identity to the polypeptide sequence as given in SEQ ID No. 1 containing a feature of residue difference corresponding to X152S and X199H, for the improved conversion of glucose to gluconic acid, with simultaneous conversion of NADP+ to NADPH.

Additionally, the engineered glucose dehydrogenase polypeptide of the present invention contains one or more of the following residue differences as compared to SEQ ID 1: The residue corresponding to X6 is glutamate, or arginine; The residue corresponding to X7 is glycine, or glutamate; The residue corresponding to X9 is valine, or arginine; The residue corresponding to X15 is serine, or alanine; The residue corresponding to X16 is serine, cysteine, threonine, or alanine; The residue corresponding to X17 is threonine, or arginine; The residue corresponding to X19 is leucine, alanine, or tyrosine; The residue corresponding to X20 is glycine, or cysteine; The residue corresponding to X21 is lysine, or histidine; The residue corresponding to X22 is serine, alanine, or lysine; The residue corresponding to X25 is isoleucine, or valine; The residue corresponding to X29 is threonine, arginine, lysine, or alanine; The residue corresponding to X31 is lysine, glutamine, or asparagine; The residue corresponding to X33 is lysine, aspartate, arginine, or glutamine; The residue corresponding to X36 is valine, or arginine; The residue corresponding to X38 is tyrosine, or cysteine; The residue corresponding to X40 is serine, leucine, or glutamate; The residue corresponding to X41 is lysine, or arginine; The residue corresponding to X41 is lysine, or glutamate; The residue corresponding to X42 is glutamate, lysine, or glutamine; The residue corresponding to X45 is alanine, or aspartate; The residue corresponding to X46 is asparagine, or aspartate; The residue corresponding to X47 is serine, aspartate, or lysine; The residue corresponding to X49 is leucine, or valine; The residue corresponding to X53 is lysine, or histidine; The residue corresponding to X56 is glycine, asparagine, serine, or aspartate; The residue corresponding to X57 is glycine, lysine, aspartate, proline, or asparagine; The residue corresponding to X58 is glutamate, lysine, or isoleucine; The residue corresponding to X60 is isoleucine, or arginine; The residue corresponding to X61 is alanine, lysine, or arginine; The residue corresponding to X62 is valine, or aspartate; The residue corresponding to X73 is isoleucine, or lysine; The residue corresponding to X74 is asparagine, or arginine; The residue corresponding to X78 is serine, glutamate, or lysine; The residue corresponding to X83 is phenylalanine, or aspartate; The residue corresponding to X83 is phenylalanine, or glutamate; The residue corresponding to X92 is asparagine, or cysteine; The residue corresponding to X95 is leucine, or isoleucine; The residue corresponding to X96 is glutamate, glutamine, valine, aspartate, alanine, isoleucine, or methionine; The residue corresponding to X97 is asparagine, or isoleucine, valine; The residue corresponding to X98 is proline, tyrosine, phenylalanine, threonine, asparagine, alanine, or serine; The residue corresponding to X100 is serine, threonine, alanine, or proline; The residue corresponding to X101 is serine, threonine, or alanine; The residue corresponding to X102 is histidine, or lysine; The residue corresponding to X105 is serine, lysine, or threonine; The residue corresponding to X107 is serine, or glutamate; The residue corresponding to X108 is aspartate, glutamate, or leucine; The residue corresponding to X110 is asparagine, arginine, or histidine; The residue corresponding to X113 is isoleucine, or aspartate; The residue corresponding to X117 is leucine, or tyrosine; The residue corresponding to X118 is threonine, lysine, arginine, or glutamate; The residue corresponding to X120 is alanine, or threonine; The residue corresponding to X122 is leucine, or glutamate; The residue corresponding to X131 is phenylalanine, or cysteine; The residue corresponding to X132 is valine, or aspartate; The residue corresponding to X137 is lysine, or cysteine; The residue corresponding to X138 is glycine, or cysteine; The residue corresponding to X139 is threonine, or aspartate; The residue corresponding to X146 is valine, aspartate, serine, alanine, isoleucine, or glutamate; The residue corresponding to X147 is histidine, serine, alanine, tyrosine, proline, arginine, glutamine, isoleucine, valine, asparagine, glycine, phenylalanine, threonine, or glutamate; The residue corresponding to X148 is glutamate, or cysteine; The residue corresponding to X149 is lysine, glutamate, threonine, or isoleucine; The residue corresponding to X151 is proline, valine, tyrosine, phenylalanine, alanine, aspartate, methionine, cysteine, glutamate, histidine, or serine; The residue corresponding to X153 is proline, methionine, asparagine, threonine, leucine, alanine, cysteine, or isoleucine; The residue corresponding to X154 is leucine, valine, tryptophan, glutamine, threonine, or asparagine; The residue corresponding to X155 is phenylalanine, aspartate, asparagine, isoleucine, proline, leucine, valine, serine, threonine, histidine, tryptophan, methionine, glutamine, glutamate, or cysteine; The residue corresponding to X160 is alanine, cysteine, or lysine; The residue corresponding to X163 is glycine, or alanine; The residue corresponding to X164 is glycine, or cysteine; The residue corresponding to X166 is lysine, arginine, or cysteine; The residue corresponding to X167 is leucine, or lysine; The residue corresponding to X168 is methionine, or cysteine; The residue corresponding to X170 is glutamate, or lysine; The residue corresponding to X175 is glutamate, or cysteine; The residue corresponding to X177 is alanine, cysteine, or aspartate; The residue corresponding to X179 is lysine, or arginine; The residue corresponding to X180 is glycine, cysteine, serine, or glutamate; The residue corresponding to X185 is asparagine, leucine, or glutamine; The residue corresponding to X187 is glycine, or alanine; The residue corresponding to X189 is glycine, lysine, glutamate, cysteine, aspartate, threonine, or alanine; The residue corresponding to X190 is alanine, cysteine, proline, or glycine; The residue corresponding to X191 is isoleucine, leucine, phenylalanine, serine, histidine, proline, tyrosine, methionine, or glycine; The residue corresponding to X192 is asparagine, aspartate, or arginine; The residue corresponding to X194 is proline, alanine, glutamine, valine, glutamate, methionine, histidine, or phenylalanine; The residue corresponding to X195 is isoleucine, glutamate, tryptophan, glycine, serine, valine, alanine, threonine, proline, histidine, aspartate, arginine, asparagine, glutamine, tyrosine, lysine, or methionine; The residue corresponding to X196 is asparagine, glutamate, threonine, or alanine; The residue corresponding to X197 is alanine, valine, tryptophan, histidine, asparagine, lysine, or isoleucine; The residue corresponding to X198 is glutamate, tyrosine, cysteine, histidine, valine, leucine, arginine, isoleucine, glycine, serine, methionine, asparagine, threonine, glutamine, phenylalanine, tryptophan, alanine, or aspartate; The residue corresponding to X203 is proline, alanine, or phenylalanine; The residue corresponding to X204 is glutamate, valine, glutamine, lysine, or alanine; The residue corresponding to X205 is glutamine, lysine, or arginine; The residue corresponding to X207 is alanine, asparagine, lysine, arginine, or serine; The residue corresponding to X208 is aspartate, glutamate, glycine, or lysine; The residue corresponding to X209 is valine, or threonine; The residue corresponding to X211 is serine, alanine, glutamate, glutamine, leucine, or methionine; The residue corresponding to X212 is methionine, leucine, or threonine; The residue corresponding to X214 is proline, or cysteine; The residue corresponding to X215 is methionine, cysteine, leucine, or glutamate; The residue corresponding to X216 is glycine, arginine, or valine; The residue corresponding to X217 is tyrosine, valine, or arginine; The residue corresponding to X218 is isoleucine, or aspartate; The residue corresponding to X220 is glutamate, or arginine; The residue corresponding to X222 is glutamate, lysine, or arginine; The residue corresponding to X223 is glutamate, or cysteine; The residue corresponding to X227 is valine, or lysine; The residue corresponding to X230 is tryptophan, phenylalanine, or tyrosine; The residue corresponding to X234 is serine, lysine, aspartate, or glutamate; The residue corresponding to X235 is glutamate, or arginine; The residue corresponding to X237 is serine, histidine, lysine, glutamate, arginine, or alanine; The residue corresponding to X238 is tyrosine, or cysteine; The residue corresponding to X240 is threonine, or lysine; The residue corresponding to X242 is isoleucine, glutamine, lysine, or glutamate; The residue corresponding to X243 is threonine, alanine, glycine, or lysine; The residue corresponding to X244 is leucine, isoleucine, or aspartate; The residue corresponding to X248 is glycine, cysteine, or lysine; The residue corresponding to X250 is methionine, isoleucine, asparagine, aspartate, serine, glycine, threonine, alanine, glutamate, cysteine, tryptophan, proline, or leucine; The residue corresponding to X252 is glutamine, or lysine; The residue corresponding to X253 is tyrosine, or cysteine; The residue corresponding to X255 is serine, cysteine, leucine, tyrosine, phenylalanine, histidine, glycine, glutamate, glutamine, alanine, or aspartate; The residue corresponding to X256 is phenylalanine, proline, glutamine, histidine, leucine, alanine, tryptophan, or arginine; The residue corresponding to X257 is glutamine, phenylalanine, alanine, cysteine, tyrosine, lysine, leucine, or methionine; The residue corresponding to X258 is alanine, arginine, tryptophan, glutamate, asparagine, lysine, valine, tryptophan, glutamate, asparagine, lysine, or valine;

In some embodiments, the engineered glucose dehydrogenase polypeptide given in SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, and 30 can have an amino acid difference by one or more of the above mentioned substitutions in combination with one or multiple residue differences when compared to SEQ ID NO: 1 (Table 3)

### Advantages/Significance of the Invention

The invention offers a significant advantage by eliminating the need for traditional spatial alignment. By focusing on the chemical composition and atomic-level properties, the method can compare proteins without requiring superimposition or matching of their overall structures. This enables more accurate comparison of proteins with divergent overall shapes, allowing the identification of functionally relevant regions even in proteins with low global similarity.

The invention provides a computational approach that can identify high-energy residues and functional regions within proteins by analyzing their potential energy distribution. This leads to better predictions of protein function and the identification of critical sites for mutation or modification.

By capturing the localized atomic properties in a protein's structure through a grid-based approach, this method enables the precise engineering of proteins, antibodies, and enzymes. It allows for targeted modifications that enhance the stability, activity, and specificity of proteins for therapeutic, and industrial purposes.

The use of localized spherical feature grids (LSFGs) and advanced machine learning models (e.g., transformer-based similarity method) enables the analysis of a wide range of proteins, regardless of their structural differences. This versatility makes the invention applicable to numerous areas, including antibody engineering, enzyme engineering, and functional domain identification.

What is claimed is:

1. A method for engineering proteins with desired functionalities, comprising:
a. A localized spherical feature grid is constructed for a protein of interest by defining a three-dimensional grid around specific regions with a 6.0 Å radius and grid points uniformly spaced at 1.0 Å intervals.
b. Assigning atomic descriptors to each grid point based on the atomic properties within proximity, wherein the descriptors include atom type, partial charge, polarity, atomic volume, solvent access surface area, solvent accessibility, electronegativity, ionization energy, polarizability, electron affinity, electrostatic potential, and coordination number in combination;
c. Calculating composite atomic properties for overlapping atoms at grid points using weighted aggregation methods to accurately reflect chemical and spatial characteristics;
d. Comparing the LSFG of the protein of interest with a database of predefined LSFGs derived from proteins with known functionalities, wherein the comparison includes geometric alignment using rotation matrices and quaternions to evaluate spatial alignment through Euclidean distance and/or cosine similarity metrics in combination to derive a combined score for LSFG comparison;
e. Identifying regions of high similarity between the LSFG of the protein of interest and the predefined LSFGs to predict structural and functional attributes of the protein of interest;
f. Engineering the protein of interest by introducing mutations in the localized regions identified through LSFG matching to enhance desired properties.

2. The method of claim 1, wherein the specific region of the protein is determined using a grid-based approach comprising of steps:
a. Creating a three-dimensional grid around the three dimensional structure of the protein of interest, wherein the grid construction includes defining a spatial arrangement that encloses the entire protein and setting grid points at regular intervals of 0.5 Å to ensure high-resolution coverage.
b. Placing probe atoms, including carbon, nitrogen, oxygen, sulphur, and hydrogen, at each grid point to assess the energy landscape across the protein, wherein potential energy values are calculated at each probe atom to generate an energy map of the protein.
c. The process involves mapping energy values onto a three-dimensional grid constructed around the protein, sorting the mapped energy values, and identifying residues corresponding to high-energy regions.

3. The method of claim 1, wherein the predefined LSFGs in the database are derived from proteins with characteristics selected from the group consisting of thermostability, pH tolerance, organic solvent tolerance, and functional domain activity.

4. The method of claim 1, wherein the LSFG comparison step is enhanced by A Machine-learning-based analysis to evaluate spatial and chemical similarity through embedded grid point tokens that uses a scoring system based on transformer attention mechanisms to identify key residues contributing to functional similarities.

5. The method of claim 1, wherein the proximity of atoms to equidistant grid points is determined based on the proximity of other bonded atoms to either of the grid points.

6. The method of claim 1, further comprising cloning the engineered protein into an expression vector, expressing the protein in a suitable host organism, and validating its catalytic efficiency in a target reaction.

7. The method of claim 1, wherein the protein of interest is an enzyme, specifically, a glucose dehydrogenase enzyme wherein the engineered glucose dehydrogenase protein comprises a sequence at least 90% identical to SEQ ID NO: 1 and contains mutations at residues corresponding to X152S and X199H, and the LSFG is used to optimize residues involved in substrate binding, cofactor recycling, or active site stabilization, enhancing its activity in glucose-to-gluconic acid conversion while recycling NADP+ to NADPH.

8. The engineered glucose dehydrogenase (GDH) enzyme as claimed in claim 7, where, the engineered glucose dehydrogenase polypeptides given in SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, and 30 can have an amino acid difference by one or more of the following substitutions, in combination with one or multiple residue differences when compared to SEQ ID NO:1, wherein the residues confer enhanced structural stability and catalytic efficiency:
The residue corresponding to X7 is glycine, or glutamate;
The residue corresponding to X9 is valine, or arginine;
The residue corresponding to X15 is serine, or alanine;
The residue corresponding to X16 is serine, cysteine, threonine, or alanine;
The residue corresponding to X17 is threonine, or arginine;
The residue corresponding to X19 is leucine, alanine, or tyrosine;
The residue corresponding to X20 is glycine, or cysteine;
The residue corresponding to X21 is lysine, or histidine;
The residue corresponding to X22 is serine, alanine, or lysine;
The residue corresponding to X25 is isoleucine, or valine;
The residue corresponding to X29 is threonine, arginine, lysine, or alanine;
The residue corresponding to X31 is lysine, glutamine, or asparagine;
The residue corresponding to X33 is lysine, aspartate, arginine, or glutamine;
The residue corresponding to X36 is valine, or arginine;
The residue corresponding to X38 is tyrosine, or cysteine;
The residue corresponding to X40 is serine, leucine, or glutamate;
The residue corresponding to X41 is lysine, or arginine;
The residue corresponding to X41 is lysine, or glutamate;
The residue corresponding to X42 is glutamate, lysine, or glutamine;
The residue corresponding to X45 is alanine, or aspartate;
The residue corresponding to X46 is asparagine, or aspartate;
The residue corresponding to X47 is serine, aspartate, or lysine;
The residue corresponding to X49 is leucine, or valine;
The residue corresponding to X53 is lysine, or histidine;
The residue corresponding to X56 is glycine, asparagine, serine, or aspartate;
The residue corresponding to X57 is glycine, lysine, aspartate, proline, or asparagine;
The residue corresponding to X58 is glutamate, lysine, or isoleucine;
The residue corresponding to X60 is isoleucine, or arginine;
The residue corresponding to X61 is alanine, lysine, or arginine;
The residue corresponding to X62 is valine, or aspartate;
The residue corresponding to X73 is isoleucine, or lysine;
The residue corresponding to X74 is asparagine, or arginine;
The residue corresponding to X78 is serine, glutamate, or lysine;
The residue corresponding to X83 is phenylalanine, or aspartate;
The residue corresponding to X83 is phenylalanine, or glutamate;
The residue corresponding to X92 is asparagine, or cysteine;
The residue corresponding to X95 is leucine, or isoleucine;
The residue corresponding to X96 is glutamate, glutamine, valine, aspartate, alanine, isoleucine, or methionine;
The residue corresponding to X97 is asparagine, or isoleucine, valine;
The residue corresponding to X98 is proline, tyrosine, phenylalanine, threonine, asparagine, alanine, or serine;
The residue corresponding to X100 is serine, threonine, alanine, or proline;
The residue corresponding to X101 is serine, threonine, or alanine;
The residue corresponding to X102 is histidine, or lysine;
The residue corresponding to X105 is serine, lysine, or threonine;
The residue corresponding to X107 is serine, or glutamate;
The residue corresponding to X108 is aspartate, glutamate, or leucine;
The residue corresponding to X110 is asparagine, arginine, or histidine;
The residue corresponding to X113 is isoleucine, or aspartate;
The residue corresponding to X117 is leucine, or tyrosine;
The residue corresponding to X118 is threonine, lysine, arginine, or glutamate;
The residue corresponding to X120 is alanine, or threonine;
The residue corresponding to X122 is leucine, or glutamate;
The residue corresponding to X131 is phenylalanine, or cysteine;
The residue corresponding to X132 is valine, or aspartate;
The residue corresponding to X137 is lysine, or cysteine;
The residue corresponding to X138 is glycine, or cysteine;
The residue corresponding to X139 is threonine, or aspartate;
The residue corresponding to X146 is valine, aspartate, serine, alanine, isoleucine, or glutamate;
The residue corresponding to X147 is histidine, serine, alanine, tyrosine, proline, arginine, glutamine, isoleucine, valine, asparagine, glycine, phenylalanine, threonine, or glutamate;
The residue corresponding to X148 is glutamate, or cysteine;
The residue corresponding to X149 is lysine, glutamate, threonine, or isoleucine;
The residue corresponding to X151 is proline, valine, tyrosine, phenylalanine, alanine, aspartate, methionine, cysteine, glutamate, histidine, or serine;
The residue corresponding to X153 is proline, methionine, asparagine, threonine, leucine, alanine, cysteine, or isoleucine;
The residue corresponding to X154 is leucine, valine, tryptophan, glutamine, threonine, or asparagine;
The residue corresponding to X155 is phenylalanine, aspartate, asparagine, isoleucine, proline, leucine, valine, serine, threonine, histidine, tryptophan, methionine, glutamine, glutamate, or cysteine;
The residue corresponding to X160 is alanine, cysteine, or lysine;
The residue corresponding to X163 is glycine, or alanine;
The residue corresponding to X164 is glycine, or cysteine;
The residue corresponding to X166 is lysine, arginine, or cysteine;
The residue corresponding to X167 is leucine, or lysine;
The residue corresponding to X168 is methionine, or cysteine;
The residue corresponding to X170 is glutamate, or lysine;
The residue corresponding to X175 is glutamate, or cysteine;
The residue corresponding to X177 is alanine, cysteine, or aspartate;
The residue corresponding to X179 is lysine, or arginine;
The residue corresponding to X180 is glycine, cysteine, serine, or glutamate;
The residue corresponding to X185 is asparagine, leucine, or glutamine;
The residue corresponding to X187 is glycine, or alanine;
The residue corresponding to X189 is glycine, lysine, glutamate, cysteine, aspartate, threonine, or alanine;
The residue corresponding to X190 is alanine, cysteine, proline, or glycine;
The residue corresponding to X191 is isoleucine, leucine, phenylalanine, serine, histidine, proline, tyrosine, methionine, or glycine;
The residue corresponding to X192 is asparagine, aspartate, or arginine;
The residue corresponding to X194 is proline, alanine, glutamine, valine, glutamate, methionine, histidine, or phenylalanine;
The residue corresponding to X195 is isoleucine, glutamate, tryptophan, glycine, serine, valine, alanine, threonine, proline, histidine, aspartate, arginine, asparagine, glutamine, tyrosine, lysine, or methionine;
The residue corresponding to X196 is asparagine, glutamate, threonine, or alanine;
The residue corresponding to X197 is alanine, valine, tryptophan, histidine, asparagine, lysine, or isoleucine;
The residue corresponding to X198 is glutamate, tyrosine, cysteine, histidine, valine, leucine, arginine, isoleucine, glycine, serine, methionine, asparagine, threonine, glutamine, phenylalanine, tryptophan, alanine, or aspartate;
The residue corresponding to X203 is proline, alanine, or phenylalanine;
The residue corresponding to X204 is glutamate, valine, glutamine, lysine, or alanine;
The residue corresponding to X205 is glutamine, lysine, or arginine;
The residue corresponding to X207 is alanine, asparagine, lysine, arginine, or serine;
The residue corresponding to X208 is aspartate, glutamate, glycine, or lysine;
The residue corresponding to X209 is valine, or threonine;
The residue corresponding to X211 is serine, alanine, glutamate, glutamine, leucine, or methionine;
The residue corresponding to X212 is methionine, leucine, or threonine;
The residue corresponding to X214 is proline, or cysteine;
The residue corresponding to X215 is methionine, cysteine, leucine, or glutamate;
The residue corresponding to X216 is glycine, arginine, or valine;
The residue corresponding to X217 is tyrosine, valine, or arginine;
The residue corresponding to X218 is isoleucine, or aspartate;
The residue corresponding to X220 is glutamate, or arginine;
The residue corresponding to X222 is glutamate, lysine, or arginine;
The residue corresponding to X223 is glutamate, or cysteine;
The residue corresponding to X227 is valine, or lysine;
The residue corresponding to X230 is tryptophan, phenylalanine, or tyrosine;
The residue corresponding to X234 is serine, lysine, aspartate, or glutamate;
The residue corresponding to X235 is glutamate, or arginine;
The residue corresponding to X237 is serine, histidine, lysine, glutamate, arginine, or alanine;
The residue corresponding to X238 is tyrosine, or cysteine;
The residue corresponding to X240 is threonine, or lysine;
The residue corresponding to X242 is isoleucine, glutamine, lysine, or glutamate;
The residue corresponding to X243 is threonine, alanine, glycine, or lysine;
The residue corresponding to X244 is leucine, isoleucine, or aspartate;
The residue corresponding to X248 is glycine, cysteine, or lysine;
The residue corresponding to X250 is methionine, isoleucine, asparagine, aspartate, serine, glycine, threonine, alanine, glutamate, cysteine, tryptophan, proline, or leucine;
The residue corresponding to X252 is glutamine, or lysine;
The residue corresponding to X253 is tyrosine, or cysteine;
The residue corresponding to X255 is serine, cysteine, leucine, tyrosine, phenylalanine, histidine, glycine, glutamate, glutamine, alanine, or aspartate;
The residue corresponding to X256 is phenylalanine, proline, glutamine, histidine, leucine, alanine, tryptophan, or arginine;
The residue corresponding to X257 is glutamine, phenylalanine, alanine, cysteine, tyrosine, lysine, leucine, or methionine;
The residue corresponding to X258 is alanine, arginine, tryptophan, glutamate, asparagine, lysine, valine, tryptophan, glutamate, asparagine, lysine, or valine.

9. The method of claim 1, further comprising of:
a. designing antibodies with enhanced binding specificity and affinity, wherein LSFGs are used to identify critical residues in antigen-binding domains.
b. predicting novel protein functions or annotate uncharacterized proteins through structural and chemical comparisons using the identified functional domains.

10. The method of claim 1, wherein the engineered protein exhibits improved activity in conditions of elevated temperature, extreme pH, or organic solvents.


SEQUENCE LISTING (30 sequences)

SEQ ID NO: 1 - protein, 261 aa, synthetic construct
MYKDLEGKVVVITGSSTGLGKSMAIRFATEKAKVVVNYRSKEDEANSVLEEIKKVGGEAIAVKGDVTVESDVINLVQSAIKEFGKLDVMINNAGLENPVSSHEMSLSDWNKVIDTNLTGAFLGSREAIKYFVENDIKGTVINMSSVHEKIPSPLFVHYAASKGGMKLMTETLALEYAPKGIRVNNIGPGAINTPINAEHFADPEQRADVESMIPMGYIGEPEEIAAVAAWLASSEASYVTGITLFADGGMTQYPSFQAGRG

SEQ ID NO: 2 - protein, 261 aa, synthetic construct
MYKDLEGKVVVITGSSTGLGKSMAIRFATEKAKVVVNYRSKEDEANSVLEEIKKVGGEAIAVKGDVTVESDVINLVQSAIKEFGKLDVMINNAGLENPVSSHEMSLSDWNKVIDTNLTGAFLGSREAIKYFVENDIKGTVINMSSVHEKIPSPLFVHYAASKGGMKLMTETLALEYAPKGIRVNNIGPGAINTPINAEHFADPEQRADVESMIPMGYIGEPEEIAAVAAFLASSEASYVTGITLFADGGMTQYPSFQAGRG

SEQ ID NO: 3 - protein, 261 aa, synthetic construct
MYKDLEEKRVVITGSSTGLGKSMAIRFATEKAKVVVNYRSKEDEANSVLEEIKKVGGEAIAVKGDVTVESDVINLVQSAIKEFGKLDVMINNAGLENPVSSHEMSLSDWNKVIDTNLTGAFLGSREAIKYFVENDIKGTVINMSSVHEKIPSPLFVHYAASKGGMKLCTKTLALEYAPKGIRVNNIGPGAINTPINAEHFADPEQRADVESTIPMGYIGEPEEIAAVAAWLASSEASYVTGITLFADGGMTQYPSFQAGRG

SEQ ID NO: 4 - protein, 261 aa, synthetic construct
MYKDLEGKVVVITGSSTGLGKSMAIRFATEKAKVVVNYRSKKDEANSVLEEIKKVGGEAIAVKGDVTVESDVINLVQSAIKEFGKLDVMINNAGLENPVSSHEMSLSDWNKVIDTNLTGAFLGSREAIKYFVENDIKGTVINMSSVHEKIPSPLFVHYAASKGGMKLCTETLALCYAPKGIRVNNIGPGAINTPINAEHFADPEQRADVESMIPMGYIGEPEEIAAVAAWLASSEASYVTGITLFADGGMTQYPQFQAGRG

SEQ ID NO: 5 - protein, 261 aa, synthetic construct
MYKDLEGKVVVITGSSTGLGKSMAIRFATEKAKVVVNYRSKEDEANSVLEEIKKVGGEAIAVKGDVTVESDVINLVQSAIKEFGKLDVMINNAGLENPVSAHEMSLSDWHKVIDTNLKGAFLGSREAIKYFVENDIKGTVINMSSVHEKIPSPLFVHYAASKGGMKLMTETLALEYAPKGIRVNNIGPGAINTPINAEHFADPEQRADVESMIPMRYIGEPEEIAAVAAWLASSEASYVTGITLFADGGMTQYPSFQAGRG

SEQ ID NO: 6 - protein, 261 aa, synthetic construct
MYKDLEGKVVVITGSSTGLGKSMAIRFATEKAKVVVNYRSKEDEANSVLEEIKKVGGEARAVKGDVTVESDVINLVQSAIKEFGKLDVMINNAGLENPVSSHEMSLSDWNKVIDTNYTGAFLGSREAIKYFVENDIKGTVINMSSVHEKIPSPLFVHYAASKGGMKLMTETLALEYAPKGIRVNNIGPGAINTPINAEHFADPEQRADVESMIPMGYIGEPEEIAAVAAWLASDEASYVTGITLFADGGMTQYPSFQAGRG

SEQ ID NO: 7 - protein, 261 aa, synthetic construct
MYKDLEGKVVVITGSSTGLGKSMAIRFATEKAKVVVNYRSKEDEANSVLEEIKKVGGEAIAVKGDVTVESDVIRLVQSAIKEFGKLDVMINNAGLENPVSSHEMSLSDWNKVDDTNLTGAFLGSREAIKYFVENDIKGTVINMSSVHEKIPSCLQVHYAASKGGMKLMTETLALEYAPKGIRVNNIGPGAIRTPINAEHFADPEQRADVESMIPMGYIGEPEEIAAVAAWLASKEASYVTGITLFADGGMTQYPSFQAGRG

SEQ ID NO: 8 - protein, 261 aa, synthetic construct
MYKDLEGKVVVITGSSTGLGKSMAIRFAREKAKVVVNYRSKKDEANSVLEEIKKVGGEAIAVKGDVTVESDVINLVQSAIKEFGKLDVMINNAGLENPVSSHEMSLSDWNKVIDTNLTGAFLGSREAIKYFVENDIKGTVINMSSEIEKIPSPLFVHYAASKGGMKLMTETLALEYAPKGIRVNNIGPGAINTPINAEHFADPEQRADVESMIPMGYIGEPEEIAAVAAWLASSEASYVTGITLFADGGMTQYPSFQAGRG

SEQ ID NO: 9 - protein, 261 aa, synthetic construct
MYKDLEGKVVVITGSSTGLGKSMAIRFATEKAKVVVNYRSKEDEANSVLEEIKKVSGEAIAVKGDVTVESDVINLVQSAIKEFGKLDVMINNAGLENPVSSHEMSLSDWNKVIDTNLTGAFLGSREAIKYFVENDIKGTVINMSSSHEKIPSPLNVHYAASKGGMKLMTETLALEYAPKGIRVNNIGPGAINTPINAEHFADPEQRADVESMIPMGYIGEPEEIAAVAAWLASSEASYVTGITLFADGGMTQYPSFQAGRG

SEQ ID NO: 10 - protein, 261 aa, synthetic construct
MYKDLEGKVVVITGSCTGLGKSMAIRFATEKAKVVVNYRSKEDEANSVLEEIKKVGGEAIAVKGDVTVESDVINLVQSAIKEFGKLDVMINNAGLENPVSSHEMSLSDWNKVIDTNLTGAFLGSREAIKYFVENDIKGTVINMSSVFEKIPSPLFVHYAASKGGMCLMTETLALEYCPKGIRVNNIGPGAINTPINAEHFADPEQRADVESTIPMGYIGRPEEIAAVAAWLASSEASYVTGITLFADGGMTQYPSFQAGRG

SEQ ID NO: 11 - protein, 261 aa, synthetic construct
MYKDLEGKVVVITGSSTGLGKSMAVRFATEKAKVVVNYRSKEDEANSVLEEIKKVGGEAIAVKGDVTVESDVINLVQSAIKEFGKLDVMINNAGLENPVSSHEMSLSDWNKVIDTNLTGAFLGSREAIKYFVENDIKGDVINMSSVHEKIPSPLFVHYAASKGGMCLMTETLALEYAPKGIRVNNIGPKAINTPINAEHFADFEQRADVESMIPMGYIGEPEEIAAVAAWLASSEASYVTGITLFADGGMTQYPSFQAGRG

SEQ ID NO: 12 - protein, 261 aa, synthetic construct
MYKDLEGKVVVITGSSTGLGKSMAIRFATEKAKVVVNYRSKEDEANSVLEEIHKVGGEAIAVKGDVTVESDVINLVQSAIKEFGKLDVMINNAGLENPVSSHEMSLSDWNKVIDTNLTGAFLGSREAIKYFVENDIKGTVINMSSVYEKIPSPLFVHYAASKAGMKKMTETLALEYAPKGIRVNNIGPGAINTPINAEHFADPEQRADVESMIPMGYIGEPEEIAAVAAWLASSEASYVTGITLFADGGMTQYPSFQAGRG

SEQ ID NO: 13 - protein, 261 aa, synthetic construct
MYKDLEGKVVVITGSSTGLGHSMAIRFATEKAKVVVNYRSKEDEANSVLEEIKKVGDEAIAVKGDVTVESDVINLVQSAIKEFGKLDVMINNAGLENPVSSHEMSLSDWNKVIDTNYTGAFLGSREAIKYFVENDIKCTVINMSSVHEKIASPLFVHYAASKGGMKLMTKTLALEYAPKGIRVNNIGPGAINTPIEAEHFADPEQRADVEEMIPMGYIGEPEEIAAVAAWLASSEASYVTGITLFADGGMTQYPSFQAGRG

SEQ ID NO: 14 - protein, 261 aa, synthetic construct
MYKDLEGKVVVITGSSTGLGKSMAIRFATEKAKVVVNYRSKEDEANSVLEEIKKVGGEAIAVKGDVTVESDVINLVQSAIKEFGKLDVMINNAGLENPVSSHEMSLSDWNKVIDTNLTGAFLGSREAIKYFVENDIKGTVINMSSVIEIIPSCLFVHYAASKGGMKLMTETLALEYAPKGIRVNNIGPGAFNTPINAEHFADPEQRADVESMIPMGYIGEPEEIAAVAAWLASSEASYVTGITDFADCGMTQYPSFQAGRG

SEQ ID NO: 15 - protein, 261 aa, synthetic construct
MYKDLEGKVVVITGSSTGLGKSMAIRFATEKAKVVVNYRSKEDEANSVLEEIKKVGGEAIAVKGDVTVESDVINLVQSAIKEFGKLDVMINNAGLENPVSSHEMSLSDWNKVIDTNYTGAFLGSREAIKYFVENDIKCTVINMSSVHEKIPSPLPVHYAASKGGMKLMTETLALEYCPKGIRVNNIGPGAINTPINAEHFADPEQRADVESMIPMGVIGRPEEIAAVAAWLASSEASYVTGITLFADGGMTQYPLFQAGRG

SEQ ID NO: 16 - protein, 261 aa, synthetic construct
MYKDLEGKVVVITGSSTGLGKSMAIRFATEKAKVVVNYRSKEDEANSVLEEIKKVGGEAIAVKGDVTVESDVINLVQSAIKEFGKLDVMINNAGLENPVSSHEMSLSDWNKVDDTNLKGTFLGSREAIKYFVENDIKGTVINMSSIHEKIPSPLFVHYAASKGGMKLMTETLALEYCPKGIRVNNIGPGAINTPINAEHFADPEQRAGVESMIPMGYIGEPEEIAAVAAWLASSEASYVTGITLFADKGMTQYPSLQAGRG

SEQ ID NO: 17 - protein, 261 aa, synthetic construct
MYKDLEGKVVVITGSSTGLGKSMAIRFATEKAKVVVNYRSKEDEANSVLEEIKKVGGEAIAVKGDVTVESDVINLVQSAIKEDGKLDVMINNAGLENPVSSHEMSLSDWRKVIDTNLTGAFEGSREAIKYFVENDIKGTVINMSSVHETIPSPLFVHYAASKAGMKLMTETLALCYAPKCIRVNNIGPGAINTPINAEHFADPEQRADVESMIPMGYDGEPEEIAAVAAYLASSEASYVTGITLFADGGMTQYPSFQAGRG

SEQ ID NO: 18 - protein, 261 aa, synthetic construct
MYKDLEGKVVVITGASTGLGKSMAIRFATEKAKVVVNYRSKEDEANSVLEEIKKVGGEAIAVKGDVTVESDVINLVQSAIKEFGKLDVMINNAGLENPVASHEMSLSDWNKVIDTNLKGAFLGSREAIKYFDENDIKGTVINMSSVHEIIPSLLMVHYAASKGGMKLMTETLALEYAPKGIRVNNIGPGAINTPINAEHFADPEQRADVESMIPMGRIGEPECIAAVAAWLASSEASYVTGITLFADGGMTQYPSFQAGRG

SEQ ID NO: 19 - protein, 261 aa, synthetic construct
MYKDLEGKVVVITGSATGLGKSMAIRFATEKAKVVVNYRSEEDEANSVLEEIKKVGGEAIAVKGDVTVESDVINLVQSAIKEFGKLDVMINNAGLENPVSSHEMSLSDWNKVIDTNLKGAFEGSREAIKYCVENDIKGTVINMSSVGEKIPSMLFVHYAASKGGMKLMTETLALEYAPKGIRVNNIAPGAINTPINAEHFADFEQRADVESMIPMGYIGEPEEIAAVAAWLASSEASYVTGITLFADGGMTQYPSWQAGRG

SEQ ID NO: 20 - protein, 261 aa, synthetic construct
MYKDLEGKVVVITGSTTGAGKSMAIRFATEKAKVVVNYRSKEDEANSVLEEIKKVGGEAIAVKGDVTVESDVINLVQSAIKEFGKLDVMINNAGLENPVSSHEMSLSDWRKVIDTNLTGAFLGSREAIKYFVENDIKCTVINMSSVFETIPSMLFVHYAASKGGMKLCTETLALEYAPKGIRVNNIGPCAINTVINAEHFADPEQRADVELMIPMGYIGEPEEIAAVAAWLASSEASYVTGITLFADGGMTQYPSFQAGRG

SEQ ID NO: 21 - protein, 261 aa, synthetic construct
MYKDLEGKVVVITGSSTGLGKSMAIRFATEKAKVVVNYRSKEDEANSVLEEIKKVGGEAIAVKGDVTVESDVINLVQEAIKEFGKLDVMINNAGLENPVSSHEMSLSDWNKVDDTNYTGAFLGSREAIKYCVENDIKGTVINMSSVHEKIPSPLFVHYAASKGCMKKCTETLALEYAPKGIRVNNIGPGAIRTPINAEHFADPEQRADVEEMIPMGYIGEPEEIAAVAAWLASSEASCVTGITLFADGGMTQYPSFQAGRG

SEQ ID NO: 22 - protein, 261 aa, synthetic construct
MYKDLEGKVVVITGSSTGLGKSMAIRFATEKAKVVVNYRSKEDEANSVLEEIKKVGGEAIAVKGDVTVESDVINLVQSAIKEFGKLDVMINNAGLMNPVSSHEMKLSDWNKVIDTNLTGAFLGSREAIKYFDENDIKGTVINMSSVICKIPSPQFVHYACSKGGMKLMTETLALEYCPRGIRVNNIGPGAINTPINAEHFADPEQRADVESMIPMGYIGEPEEIAAVAAYLASSEASYVTGITLFADGGMTQYPSFQAGRG

SEQ ID NO: 23 - protein, 261 aa, synthetic construct
MYKDLEGKVVVITGSSTGLGKSMAIRFATEKAKVVVNYRSKEDEANSVLEEIKKVGGEAIAVKGDVTVESDVINLVQSAIKEFGKLDVMINNAGLENPVSSHEMSLSDWNKVIDTNYTGTFLGSREAIKYFVENDIKGTVINMSSVHEKIVSPWFVHYAKSKGGMKLMTETLALEYAPKGIRVNNIGPGAINTPINAEHFADPEQRADVESTIPMVYIGEPEEIAAVAAWLASSEASYVTGITLFADGGATQYPAFQAGRG

SEQ ID NO: 24 - protein, 261 aa, synthetic construct
MYKDLEGKVVVITGSSTGLGKSMAIRFAREKAKVVVNYRSKEDEANSVLEEIKKVGGEAIRVKGDVTVESDVINLVQSAIKEFGKLDVMINNAGLENPVSSHEMSLEDWNKVDDTNLTGAFLGSREAIKYFVENDIKGTVINMSSDHEKIPSPLLVHYAASKGGMKLMTETLALEYAPKGIRVNNIGPGAINTPINAEHFADPEQRADVESMIPLVYIGEPEEIAAVAAWLASSRASYVTGITLFADGGMTQYPSFQAGRG

SEQ ID NO: 25 - protein, 261 aa, synthetic construct
MYKDLEGKVVVITGSSTGAGKSMAIRFATEKAKVVVNYRSKEDEANSVLEEIKKVGGEAIKVKGDVTVESDVINLVQSAIKEFGKLDVMINNAGLENPVSSHEMSLEEWNKVIDTNLTGAFLGSREAIKYFDENDIKGTVINMSSVVCKIPSPQFVHYAASKACMKLMTETLALEYAPKGIRVNLIGPGAINTPINAGHFADPEQRADVESMIPMGYIGEPEEIAAVAAWLASSEASYVTGKTLFADGGMTQYPSFQAGRG

SEQ ID NO: 26 - protein, 261 aa, synthetic construct
MYKDLRGKVVVITGSSTGLGKSMAIRFATEKAKVVVNYRSKEDEANSVLEEIKKVGNEAIAVKGDVTVESDVINLVQSAIKEFGKLDVMINNAGLENPVSSHEMSLSDWNKVIDTNLTGAFEGSREAIKYFDENDIKGDVINMSSEHEEIPSPLFVHYACSKGGMKLMTETLALEYAPKGIRVNNIGPGAINTPINAEHFADPEQRADVESMIPMGYIGEPEEIAAVAAWLASSEASYVTGETIFADGGMTQYPAFQAGRG

SEQ ID NO: 27 - protein, 261 aa, synthetic construct
MYKDLEGKRVVITGSSTGLGKSMAIRFATEKAKVVVNYRSKEDEANSVLEEIKKVGGEAIAVKGDVTVESDVINLVQSAIKEFGKLDVMINNAGLENPVSSHEMSLSDWRKVIDTNLTGAFLGSREAIKYFVENDIKGTVINMSSVGEKIPSPLFVHYAASKACMKLMTKTLALEYAPKGIRVNNIGPCAINTPINWEHFADPEQRAGVESMIPMGYIGEPEEIAAVAAWLASSEASYVTGITLFADGGMTQYPSFQAGRG

SEQ ID NO: 28 - protein, 261 aa, synthetic construct
MYKDLEGKVVVITGSSTGLGKSMAIRFATEKAKVVVNYRSKEDEANSVLEEIKKVNGEAIAVKGDVTVESDVINLVQSAIKEFGKLDVMINNAGLENPVSSHEMSLSDWHKVDDTNLTGAFLGSREAIKYFVENDICGTVINMSSAHCKIVSPLFVHYAASKGGMKLMTETLALEYAPKCIRVNNIGPGAINTPINAEHFADPEQRADVESMIPLGYDGEPEEIAAVAAWLASSEASYVTGITLFADGGMTQYPSFQAGRG

SEQ ID NO: 29 - protein, 261 aa, synthetic construct
MYKDLEGKVVVITGASTGLGHSMAIRFATEKAKVVVNYRSKEDEANSVLEEIKKVGGEAIAVKGDVTVESDVINLVQSAIKEFGKLDVMINNAGLENPVSSHEMSLSDWNKVIDTNLEGTFLGSREAIKYFVENDIKCTVINMSSVHEKIPSPLCVHYAASKAGMKLCTETLALEYDPKGIRVNNIGPGAINTPINARHFADPAQRADVESMIPMGYIGEPREIAAVAAWLASSEASYVTGITLFADGGMTQYPSFFAGRG

SEQ ID NO: 30 - protein, 261 aa, synthetic construct
MYKDLEGKVVVITGSSTGLGKSMAIRFATEKAKVVVNYRSKEDEANSVLEEIKKVGGEARAVKGDVTVESDVINLVQSAIKEFGKLDVMINNAGLENPVSSHEMSLSDWNKVIDTNLTGTFLGSREAIKYFVENDIKGDVINMSSVREKIMSPLFVHYAASKGGMRKMTKTLALEYAPKGIRVNNIAPGPINTPINAEHFADPEQRADVESMIPLGYIGEPEEIAAVAAWLASSEASYVTGITLFADGGMTQYPSFFAGRG